(19) United States
(12) Patent Application Publication
(10) Pub. No.: US 2023/0172967 A1
(43) Pub. Date: Jun. 8, 2023

(54) BETA-GLUCAN IMMUNOTHERAPIES AFFECTING THE IMMUNE MICROENVIRONMENT

(71) Applicant: HiberCell, Inc., New York, NY (US)

(72) Inventors: Nandita Bose, Plymouth, MN (US); Keith Gorden, Woodbury, MN (US); Ross Fulton, St. Paul, MN (US); Steven Leonardo, Rosemount, MN (US); Jeremy Graff, Indianapolis, IN (US)

(21) Appl. No.: 17/887,144

(22) Filed: Aug. 12, 2022

Related U.S. Application Data
(63) Continuation of application No. 17/566,133, filed on Dec. 30, 2021, now abandoned.
Application No. 17/566,133 is a division of application No. 16/068,868, filed on Jul. 9, 2018, now abandoned.
(60) Provisional application No. 62/276,667, filed on Jan. 8, 2016.

(51) Int. Cl. A61K 31/716; G01N 33/569; A61K 39/39; A61K 45/06; G01N 33/543; A61P 35/00; A61K 39/395; C07K 16/28

(52) U.S. Cl. CPC A61K 31/716; G01N 33/569; A61K 39/39; A61K 45/06; G01N 33/54366; A61P 35/00; A61K 39/39533; C07K 16/2827; C07K 16/2863; A61K 2039/55583

(57) ABSTRACT

This disclosure relates to soluble β-glucan immunotherapies that affect the tumor microenvironment. The soluble β-glucan immunotherapies promote an immunostimulatory environment, which enhances the effectiveness of the combination of anti-angiogenics and checkpoint inhibitors.

### CROSS-REFERENCE TO RELATED APPLICATION

This application claims priority to U.S. Provisional Patent Application Ser. No. 62/276,667 filed Jan. 8, 2016, which is incorporated herein by reference.

### BACKGROUND

This disclosure relates to combinations of soluble β-glucan and therapeutic agents that affect the immune microenvironment. β-glucan is a fungal PAMP and is recognized by pattern recognition molecule C3 in the serum as well as pattern recognition receptor, complement receptor 3 (CR3) on the innate immune cells, including neutrophils and monocytes. β-glucan (β(1,6)-[poly-1,3)-D-glucopyranosyl]-poly.-β(1,3)-D-glucopyranose), a polysaccharide β-glucan derived from yeast, is being developed as an immunotherapeutic agent in combination with anti-tumor monoclonal antibodies for the treatment of several cancers. β-glucan enables innate immune effector cells to kill complement-coated tumor cells through a complement CR3-dependent mechanism. Numerous animal tumor models have demonstrated that administration of soluble β-glucan in combination with a complement-activating, tumor-targeting antibody results in significantly reduced tumor growth and improved overall survival compared to either agent alone.

Cancers, however, are not just masses of malignant cells but complex “organs,” which recruit and use many other non-transformed cells. Interactions between malignant and non-transformed cells create the tumor microenvironment (TME). The non-malignant cells of the TME have a dynamic and often tumor-promoting function at various stages of carcinogenesis. A complex and dynamic network of cytokines, chemokines, growth factors, and inflammatory and matrix-remodeling enzymes drive intercellular communication within the afflicted tissue. To effectively beat cancer, therefore, therapies must be developed to suppress the tumor-promoting nature of the TME.

### SUMMARY

This disclosure describes, in one aspect, uses and compositions of soluble β-glucan immunotherapies that affect the immune microenvironment. In one embodiment, the soluble s-glucan immunotherapies may include the combination of an anti-angiogenic agent and a checkpoint inhibitor to treat cancer.

The above summary is not intended to describe each disclosed embodiment or every implementation of the technology described herein. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1A-1D. Morphologic and functional characterization of m vitro cultured human M1 and M2 macrophages.

FIG. 2A-2D. Morphological, phenotypic and functional characterization of soluble β-glucan-treated M2 macrophages.

FIG. 3A-3D. Evaluations of CD4 T cell proliferation and modulation of IFN-γ and IL-4 production in β-glucan-treated M1 and M2 macrophages from high binders and low binders.

FIG. 4. Evaluations of T cell proliferation and modulation of IFN-γ in β-glucan-treated M2 and M2a macrophages under immunosuppressive conditions.

FIG. 5A-5E. Evaluations of β-glucan on CD4/CD8 T cell proliferation and activation in the presence of Tregs.

FIG. 6A-6B. Characterization of in vitro cultured human immature monocyte-derived dendritic cells (imMoDC) and mature monocyte-derived dendritic cells (mMoDC).

FIG. 7A-7D. Evaluations of β-glucan's effect on MoDCs maturation.

FIG. 8A-8C. Results of increased CD4 T cell proliferation by M2-β-glucan due to cell-to-cell contact.

FIG. 9. Results of increased CD4 T cell proliferation by M2-β-glucan due to soluble factors.

FIG. 10. Analysis of J-glucan-treated M2 macrophages in high binders vs. low binders.

FIG. 11A-11B. Results of the functional evaluation of M2-β-glucan derived from low binders's monocytes in the presence of serum from a high binder.

FIG. 12A-12B. PD-L1 upregulation on β-glucan-treated M2 macrophages cultured in the presence of immunosuppresive cytokines (TCM).

FIG. 13. PD-L1 upregulation in MiaPaCa.

FIG. 14A-14B. Effects of soluble β-glucan on myeloid-derived suppressor cells (MDSC) FIG. 15. Evaluation of β-glucan induced PD-L1 expression on tumor cells.

FIG. 16A-16D. Results of mouse study using IMPRIME PGG in combination with DC101 antibody.

FIG. 17A-17N. In vivo effect on tumor microenvironment of soluble β-glucan and bevicizumab.

FIG. 18A-18C. Effects on the tumor microenvironment of soluble β-glucan and anti-PD-1 antibody.

FIG. 19A-19C. Results of mouse study using IMPRIME PGG in combination with anti-PD-1 antibody.

FIG. 20. Results of mouse study using IMPRIME PGG in combination with TA-99 antibody.

FIG. 21. Results of mouse study using IMPRIME PGG in combination with anti-PD-1 antibody and DC101 antibody.

### DETAILED DESCRIPTION OF ILLUSTRATIVE EMBODIMENTS

β-glucans are polymers of glucose derived from a variety of microbiological and plant sources including, for example, yeast, bacteria, algae, seaweed, mushroom, oats, and barley. Of these, yeast β-glucans have been extensively evaluated for their immunomodulatory properties. Yeast β-glucans can be present as various forms such as, for example, intact yeast, zymosan, purified whole glucan particles, solubilized zymosan polysaccharide, or highly-purified soluble β-glucans of different molecular weights. Structurally, yeast β-glucans are composed of glucose monomers organized as a β-(1,3)-linked glucopyranose backbone with periodic β-(1,3) glucopyranose branches linked to the backbone via β-(1,6) glycosidic linkages. The different forms of yeast β-glucans can function differently from one another. The mechanism through which yeast β-glucans exert their immunomodulatory effects can be influenced by the structural differences between different forms of the β-glucans such as, for example, its particulate or soluble nature, tertiary conformation, length of the main chain, length of the side chain, and frequency of the side chains. The immune stimulating functions of yeast β-glucans are also dependent upon the receptors engaged in different cell types in different species, which again, can be dependent on the structural properties of the D-glucans. In general, β-glucan immunotherapies can include administering to a subject any suitable form of β-glucan or any combination of two or more forms of β-glucan. Suitable β-glucans and the preparation of suitable β-glucans from their natural sources are described in, for example, U.S. Patent Application Publication No. US2008/0103112 A1. In some cases, the § β-glucan may be derived from a yeast such as, for example, Saccharomyces cerevisiae. In certain cases, the β-glucan may be or be derived from β(1,6)-[poly-(1,3)-D-glucopyranosyl]-poly-β(1,3)-D-glucopyranose, also referred to herein as PGG (IMPRIME PGG, Biothera, Eagan, MN), a highly purified and well characterized form of soluble yeast-derived β-glucan. Moreover, β-glucan-based immunotherapies can involve the use of, for example, a modified and/or derivatized β-glucan such as those described in International Patent Application No. PCT/US12/36795. In other cases, β-glucan immunotherapy can involve administering, for example, a particulate-soluble β-glucan or a particulate-soluble β-glucan preparation, each of which is described in, for example, U.S. Pat. No. 7,981,447.

Anticancer immunotherapeutic drugs kill cancer cells through multiple modalities; 1) direct activation of innate immune cells, 2) direct activation of adaptive immune cells, 3) indirect activation of both innate and adaptive immune cells by either making tumor cells more immunogenic or by subverting tumor-induced immunosuppression.

Myeloid cells at the tumor microenvironment (TME), including M2 macrophages, N2 neutrophils and myeloid-derived suppressor cells (MDSC), can promote immune suppression by directly causing functional exhaustion of the cytotoxic T cells or by indirectly increasing the suppressive power of T-regulatory cells (Tregs). This leads to a skewed immunostimulatory versus immunosuppressive balance in the TME. The immunostimulatory environment of the TME is largely shaped by the presence of cytotoxic T cells and NK cells, cytolytic and phagocytosis-inducing M1 macrophages, cytotoxic N1 neutrophils, humoral response inducing B cells, and antigen presenting immunogenic dendritic cells (DC). Immunostimulatory cytokines and chemokines such as interferon gamma (IFN-γ), interleukin-12 (IL-12), tumor necrosis factor-alpha (TNF-α), etc. are coordinators of the immunostimulatory activity. Immune cells that bias the immunosuppressive nature of the TME are anti-inflammatory Th2 cells. N2 neutrophils, M2 macrophages, Tregs, and tolerogenic DCs. Immunosuppressive cytokines and chemokines such as transforming growth factor-beta (TGF-β), interleukin-10 (IL-10), macrophage colony stimulating factor (M-CSF), interleukin-4 (1.1-4), etc. coordinate the immunosuppressive activity.

Soluble β-glucan, by virtue of being a pathogen associated molecular pattern (PAMP) that binds to CD11b on cells of myeloid origin, namely neutrophils and monocytes, binds and increases the immunostimulatory functions of N1 neutrophils and M1 macrophages and decreases the immunosuppressive functions of MDSCs, N2 neutrophils and M2 macrophages. This modulation leads to cross-talk between the different innate and adaptive cell-subsets in the TME and eventually tilts the balance towards immunostimulation. More specifically, once bound to peripheral blood monocytes, soluble β-glucan modulates the differentiation of monocytes to macrophages in M1/anti-tumorigenic versus M2/pro-tumorigenic polarizing conditions such that M1 polarization is enhanced which increases macrophage immunostimulatory functions and M2 polarization is inhibited which decreases macrophage immunosuppressive functions. Soluble β-glucan directly affects M2 repolarization to the M1 phenotype and drives Th1 polarization, and soluble β-glucan-primed innate immune cells generate cytokines to indirectly affect CD4 and CD8 T cell proliferation, even in the presence of Tregs, and eventually drive Th1 polarization. Thus, soluble β-glucan acts as a PAMP to enlist the full functionality of the innate immune system, to generate cross-talk with the adaptive immune system via enhanced antigen presentation and to enhance the anti-tumor efficacy of tumor-targeting antibodies, anti-angiogenic therapies, checkpoint inhibitors and combinations of these therapies.

Examples of checkpoint inhibitors which may be useful for soluble β-glucan immunotherapy include tremelimumab, ipilimumab, nivolumab, pembrolizumab, pidilizumab, atezolizumab, avelumab, durvalumab, AMP-224, BMS-936559, IMP321, lirilumab, INCB024360, NLG919, MGA271 and BMS-986016. Examples of target molecules of checkpoint inhibitors include PD-1, PD-L1, CTLA-4, IDO, B7-13 and LAG-3 Examples of anti-angiogenic agents which may be useful for soluble β-glucan immunotherapy include bevacizumab, everolimus, lenalidomide, pazopanib, ramucirumab, cetuximab, sorafenib, sunitinib, thalidomide, entinostat, thymoquinone, PHA 665752, withaferin A, Shikonin, Imiquimod, 2-methoxyestradiol, ursolic acid, WEB-2086, Combrestatin A4, Thiolutin, etc.

Soluble β-glucan elicits an adaptive immune response via the two innate cell subsets that are known to bridge innate and adaptive immune responses, monocyte-derived macrophages and dendritic cells and will upregulate PD-L1 expression on both monocyte-derived macrophages and dendritic cells. In spite of PD-L1 upregulation, soluble (β-glucan-treated monocyte-derived macrophages and dendritic cells enhance T cell activation and proliferation, and the coordinated immune response elicited by soluble β-glucan elicits a tumor response akin to adaptive immune resistance, i.e., upregulation of surface expression of PD-L1.

Soluble (β-glucan can be combined with non-complement activating, tumor-targeting immune suppression-relieving MAbs. For example, soluble β-glucan can be combined with anti-PD-L1 immune checkpoint inhibitors (Fc-engineered igG1 MAb) in the treatment of several cancers, including, melanoma, renal cell carcinoma, lung cancer, etc. The efficacy of anti-PD-1/PD-L1 antibodies can be dependent upon the expression level of PD-L1 on tumors. One of the mechanisms of PD-L1 expression on tumors is called adaptive immune resistance where PD-L1 expression is adaptively induced as a consequence of immune responses within the tumor microenvironment (e g., interferon gamma production by activated T-cells). Soluble β-glucan either directly or indirectly induces Th1 polarization. Th1s effect upregulates the expression of PD-L1 on tumor cells, and thereby enhance the anti-tumor activity of anti-PD-1/PD-L1 antibodies Examples of these checkpoint inhibitors are nivolumab and pembrolizumab.

Soluble β-glucan can be combined with non-complement activating, non-tumor targeting MAbs that may, for example, enhance immune co-stimulation. Some examples include a) anti-CD40 MAb (IgG2 MAb), targeting dendritic cells and b) anti-OX40, anti-41BB, enhancers of T-cell co-stimulation in the treatment of several cancers. These also include anti-PD-1 antibodies such as, for example, nivolumab.

Soluble β-glucan can also be combined with non-complement activating, non-tumor-targeting immune suppression-relieving small molecules and/or non-complement activating, tumor-targeting immune suppression-relieving small molecules. It can be used as an adjuvant in cancer vaccines to drive Th1 polarization.

Soluble β-glucan immunotherapies can be used therapeutically to decrease suppressive mechanisms in chronic diseases (i.e. TB) to hasten full clearance of the infection. In addition, they can be use to skew the Th2-Th1 balance in Th2-dominant autoimmune diseases (allergies, asthma, atopic diseases) to a Th1-polarized environment. Examples of agents that may be used in soluble β-glucan immunotherapies for treating chronic infectious diseases include Isoniazid, Rifampin, Ethambutol, Pyrazinamide, Although non-complement activating immune suppression-relieving agents may be preferred, especially for non-tumor targeting agents, the invention may also be carried out with complement activating immune suppression-relieving agents Examples of such agents are bavituximab, ipilimumab and tremelimumab.

When treating cancer, the soluble β-glucan immunotherapy described herein may also be combined with chemotherapeutic agents. Examples of the types of chemotherapeutic agents are alkylating agents, antimetabolites, anti-tumor antibiotics, topoisomerase inhibitors, mitotic inhibitors and corticosteroids.

The invention includes, in part, co-administering a β-glucan with another pharmaceutical agent, which, as used herein, may be an antibody preparation or a small molecule preparation or any preparation administered for affecting the TME. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that both components are simultaneously bioavailable after both are administered. Regardless of whether the components are co-administered simultaneously or sequentially, the components may be co-administered at a single site or at different sites.

In another aspect, the method includes administering to a subject a composition that includes a β-glucan moiety conjugated to an antibody, a therapeutic antibody, an anti-tumor antibody or an antibody fragment such as the Fe portion of an antibody. Modified and/or derivatized soluble β-glucan, including β-glucan conjugates of a β-glucan moiety and an antibody are described in International Patent Application No. PCT/US12/36795, which may also be applied to conjugates of antibody fragments. The β-glucan moiety may be, or be derived from a β-1,3/1,6 glucan. In this context, “derived from” acknowledges that a conjugate may necessarily be prepared by creating a covalent linkage that replaces one or more atoms of the β-glucan. As used herein, “derived from a β-1,3/1,6 glucan” refers to a portion of the β-glucan that remains as part of a conjugate after replacing one or more atoms of the β-glucan to form the covalent linkage of the conjugate.

The β-glucan, the antibody or small molecule preparation, and/or the combination of both components may be formulated in a composition along with a “carrier.” As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the (β-glucan or the antibody, its use in the therapeutic compositions is contemplated. Supplementary active ingredients also can be incorporated into the compositions.

By “pharmaceutically acceptable” is meant a material that is not biologically or otherwise undesirable, i.e., the material may be administered to an individual along with the β-glucan and/or the pharmaceutical agent without causing any undesirable biological effects or interacting in a deleterious manner with any of the other components of the pharmaceutical composition in which it is contained.

The β-glucan, the pharmaceutical agent, and/or the combination of both components may be formulated into a pharmaceutical composition. In some embodiments, the β-glucan and the pharmaceutical agent may be provided in a single formulation. In other embodiments, the p-glucan and the pharmaceutical agent may be provided in separate formulations. A pharmaceutical composition may be formulated in a variety of and/or a plurality forms adapted to one or more preferred routes of administration. Thus, a pharmaceutical composition can be administered via one or more known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition, or a portion thereof, can be administered to a mucosal surface, such as by administration to, for example, the nasal or respiratory mucosa (e.g., by spray or aerosol). A pharmaceutical composition, or a portion thereof, also can be administered via a sustained or delayed release.

A formulation may be conveniently presented in unit dosage form and may be prepared by methods well known in the art of pharmacy. Methods of preparing a composition with a pharmaceutically acceptable carrier include the step of bringing the β-glucan and/or the pharmaceutical agent into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

The β-glucan, the pharmaceutical agent, and/or the combination of both components may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

In some embodiments, the β-glucan may be derived from yeast such as, for example, Saccharomyces cerevisiae. In some embodiments, the β-glucan can include a β-1,3/1,6 glucan such as, for example, β(1,6)-[poly-(1,3)-D-glucopyranosyl]-poly-β(1,3)-D-glucopyranose.

In some embodiments, the method can include administering sufficient β-glucan to provide a dose of, for example, from about 100 ng/kg to about 50 mg/kg to the subject, although in some embodiments the methods may be performed by administering the β-glucan in a dose outside this range. In some embodiments, the method includes administering sufficient β-glucan to provide a dose of from about 10 μg/kg to about 5 mg/kg to the subject, for example, a dose of about 4 mg/kg.

Alternatively, the dose may be calculated using actual body weight obtained just prior to the beginning of a treatment course. For the dosages calculated in this way, body surface area (in²) is calculated prior to the beginning of the treatment course using the Dubois method m²=(wt kg^{0.421}×height cm^{0.725})×0.007184, In some embodiments, therefore, the method can include administering sufficient β-glucan to provide a dose of, for example, from about 0.01 mg/m² to about 10 mg/m².

In some embodiments, the method can include administering sufficient antibody that specifically binds the β-glucan to provide a dose of, for example, from about 100 ng/kg to about 50 mg/kg to the subject, although in some embodiments the methods may be performed by administering the antibody in a dose outside this range. In some embodiments, the method includes administering sufficient antibody to provide a dose of from about 10 μg/kg to about 5 mg/kg to the subject, for example, a dose of from about 100 μg/kg to about 1 mg/kg.

Alternatively, the dose may be calculated using actual body weight obtained just prior to the beginning of a treatment course. For the dosages calculated in this way, body surface area (m²) is calculated prior to the beginning of the treatment course using the Dubois method: m² (wt kg^{0.425}×height cm^{0.725})×0.007184. In some embodiments, therefore, the method can include administering sufficient antibody to provide a dose of, for example, from about 0.01 mg/m² to about 10 mg/m².

In some embodiments, the β-glucan and pharmaceutical agent may be co-administered, for example, from a single dose to multiple doses per week, although in some embodiments the method may be performed by co-administering the β-glucan and pharmaceutical agent at a frequency outside this range. In certain embodiments, the β-glucan and pharmaceutical agent may be administered from about once per year to once per week.

The term “and/or” means one or all of the listed elements or a combination of any two or more of the listed elements; the terms “comprises” and variations thereof do not have a limiting meaning where these terms appear in the description and claims; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

Establishment and characterization of in vitro cultured human M1 and M2 macrophages: CD14⁺ monocytes from human whole blood were enriched using Ficoll gradient and magnetic bead separation. Enriched monocytes (5×10⁵ cells per mL) were then cultured in either M1-polarizing (XVivo 10 media (Lonza Group) supplemented with 5% autologous serum and 100 ng/mL recombinant human granulocyte macrophage colony-stimulating factor (rhGM-CSF) (R&D Systems) or M2-polarizing (XVivo 10 media supplemented with 10% autologous serum and 50 ng/mL recombinant human macrophage colony-stimulating factor (rhM-CSF) (R&D Systems) conditions for 6 days. In experiments performed to evaluate the effect of β-glucan, whole blood was first incubated with vehicle (sodium citrate buffer) or 25 μg/mL soluble β-glucan for 2 hours at 37° C. and then the monocytes were isolated and differentiated. Morphology was checked before macrophages were harvested for phenotypic analysis Culture medium of the day 6 macrophage culture (MCM) was collected, spun down to remove contaminated cell pellet and then frozen down for subsequent cytokine analysis by ELISA or used to setup a co-culture with CD3 & CD28-stimulated CD4 T cells (MCM-CD4 T) for evaluation of either surface markers or CD4 T cell proliferation. The macrophages were used to setup a co-culture with CD3 & CD28- or CD3 only-stimulated CD4 T cells (Mac-CD4 T) on day 6 for evaluation of either surface marker modulation or effect on CD4 T cell proliferation. For Mac-CD4 T cell proliferation study, M1 or M2 macrophages were cultured with CD3 & CD28- or CD3 only-stimulated, CFSE-labeled, autologous CD4 T cells at a 1:10 ratio. T cell proliferation was measured at the end of the experiment (day 9-day 11) by flow cytometry, and results are graphically shown as CFSE-dilution peaks. The assessment of CD3-only stimulated T cells was always done on day 11. Quantitative results were reported as the Division Index (the average number of cell divisions a population underwent) calculated for each of the triplicate wells in each of the culture conditions. Culture supernatants of Mac-CD4 T cell co-cultures were collected for subsequent cytokine analysis.

For evaluation of Mac-CD4 T cell surface marker modulation, M2 macrophages were co-cultured with T cells as described above and cells were harvested on day 8, day 9 and day 10 to perform surface receptor staining on both M2 macrophages and T cells.

For MCM-CD4 T cell proliferation study, CD3 & CD28-stimulated, CFSE-labeled CD4 T cells were cultured with 50% MCM. T cell proliferation was measured on day 11 as described above. Culture supernatants of the MCM-CD4 T cell co-culture were collected for subsequent cytokine analysis. MCM-CD4 T cell surface marker evaluation was performed as described above.

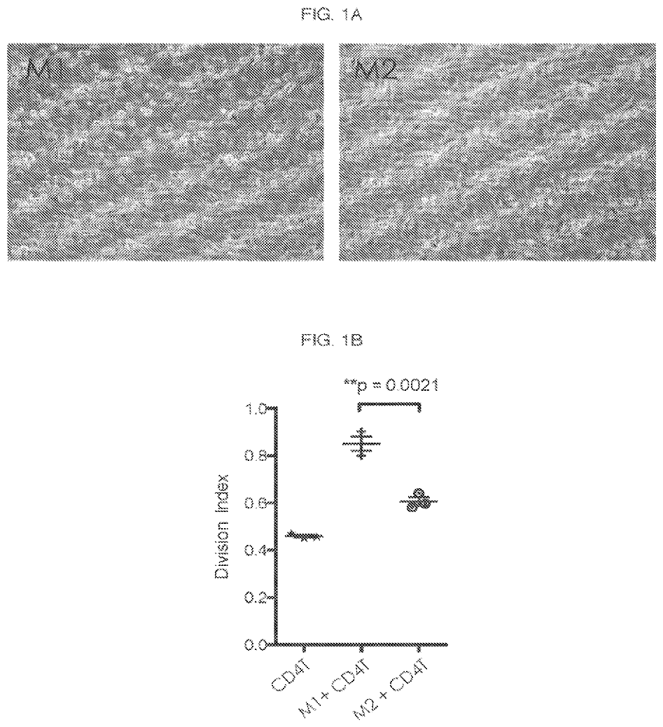

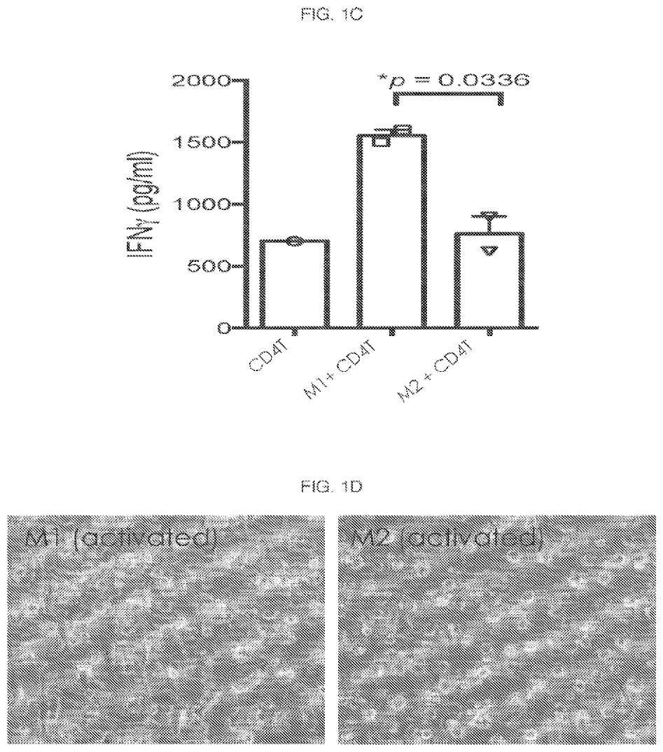

M1 and M2 macrophages were prepared and characterized as described above were characterized for A) morphology. B) phenotype. C) functional evaluation of Mac-CD4 T cell proliferation and D) cytokine analysis in the co-cultures. FIG. 1A-FIG. 1C include representative results from 5 different experiments.

As per literature, the morphology of M1 appeared more rounded and M2 were more elongated fibroblast-like (FIG. 1A). Expression of M1/M2-specific markers was evaluated by flow cytometry Median MFI was calculated for isotype control staining and surface antigen staining and the results are shown in Table 1.

TABLE 1

| | | | | | | | | | CD274 |
|---|---|---|---|---|---|---|---|---|---|
| | | HLA-DR | CD163 | CD14 | CD206 | CD209 | CD80 | CD86 | (PD-L1) |
| M1 | Isotype | 91 | 91 | 23 | 151 | 127 | 151 | 127 | 91 |
| | control | | | | | | | | |
| | Surface | 1715 | 148 | 100 | 859 | 1535 | 179 | 2218 | 3570 |
| | antigen | | | | | | | | |
| M2 | Isotype | 99 | 99 | 21 | 147 | 144 | 147 | 144 | 99 |
| | control | | | | | | | | |
| | Surface | 1054 | 1732 | 805 | 411 | 538 | 167 | 2463 | 385 |
| | antigen | | | | | | | | |

Consistent with literature regarding phenotype, M1 macrophages typically expressed higher levels of HLA-DR and CD274 (PD-L1), while M2 macrophages expressed higher levels of CD163 and CD14. Additionally, in comparison to in vitro differentiated M2 macrophages, M1 macrophages also significantly helped CD4 T cells to proliferate as shown in FIG. 1B. Concomitant with enhanced proliferation, increased production of interferon gamma (IFN-γ) was observed in the supernatants of M1 and CD4 T cell co-cultures (FIG. 1C).

Steps for an alternative method for in vitro culture and characterization of human macrophages that includes activation of M1 and M2 macrophages (designated M1a and M2a macrophages) are outlined below. Th1s methodology was used in the next series of experiments.

WB (Untreated or treated with β-glucan or vehicle for 2 hours at 37° C.)

[symbol]

Isolate PBMC by Ficoll separation

[symbol]

Enrich monocytes by negative selection

[symbol]

Culture monocytes in XVivo10 medium for 6 days:
M1-XVivo10 medium supplemented with 5% human serum &
100 ng/ml rhGM-CSF
M1a-M1 cultured medium with addition of 10 ng/ml LPS and
50 ng/ml IFN-γ
M2-XVivo10 medium supplemented with 10% human serum &
50 ng/ml rhM-CSF
M2a-M2 cultured medium with the addition of 10 ng/ml IL-4
for the last 72 hours

[symbol]

Collect macrophage culture supernatant (MCM) and harvest
cells

[symbol]

Determine morphology by microscopy
Analyze phenotype by flow cytometry
Study function of CD3/CD28- or CD3-alone stimulated T cell
proliferation in the presence of macrophages (Mac-CD4 T) by
CFSE dilution assay

Activated M1a and M2s macrophages, prepared and characterized as described above, were characterized for morphology and phenotype. Shown here are representative results from 5 different experiments.

FIG. 1D shows the morphology of M1a and M2a macrophages. Expression of M1a/M2a-specific markers was evaluated by flow cytometry. Median MFI was calculated for isotype control staining and surface antigen staining and the results are shown in Table 2

TABLE 2

| | | | | | | | | | CD274 |
|---|---|---|---|---|---|---|---|---|---|
| | | HLA-DR | CD163 | CD14 | CD206 | CD209 | CD80 | CD86 | (PD-L1) |
| M1a | Isotype | 123 | 123 | 23 | 119 | 79 | 119 | 79 | 23 |
| | control | | | | | | | | |
| | Surface | 1535 | 812 | 191 | 1005 | 1215 | 537 | 3187 | 26135 |
| | antigen | | | | | | | | |
| M2a | Isotype | 85 | 85 | 25 | 112 | 97 | 112 | 97 | 85 |
| | control | | | | | | | | |
| | Surface | 1800 | 3509 | 614 | 2186 | 4482 | 230 | 2445 | 4471 |
| | antigen | | | | | | | | |

### Example 2

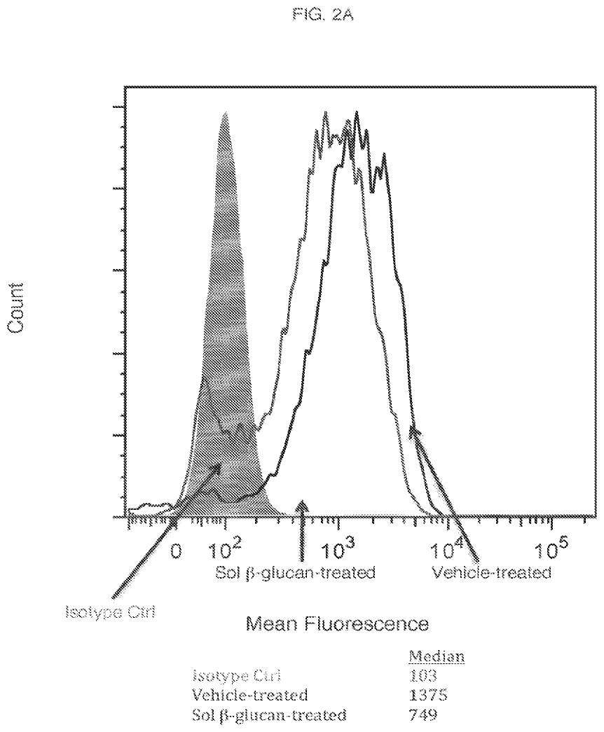

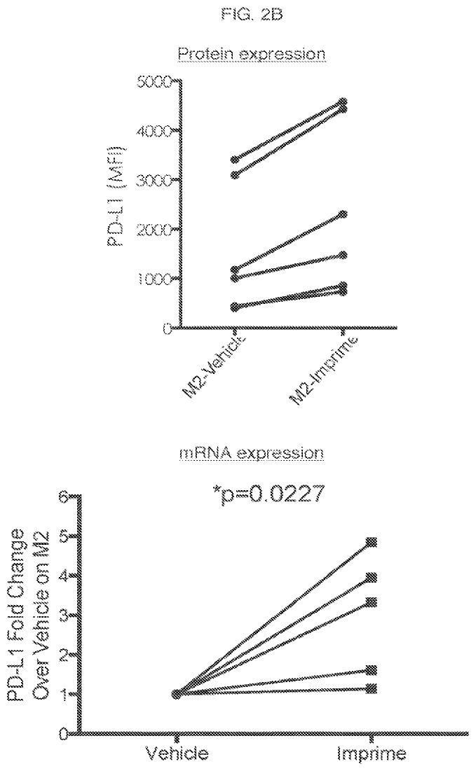

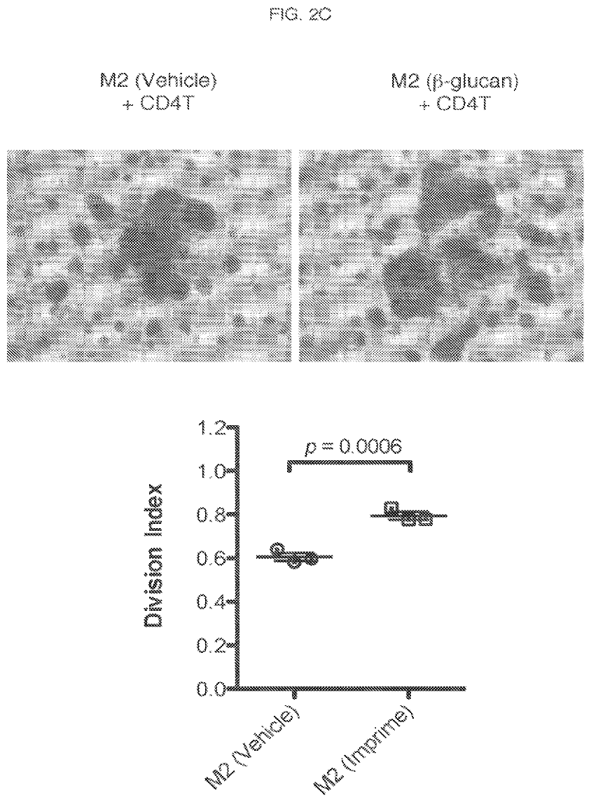

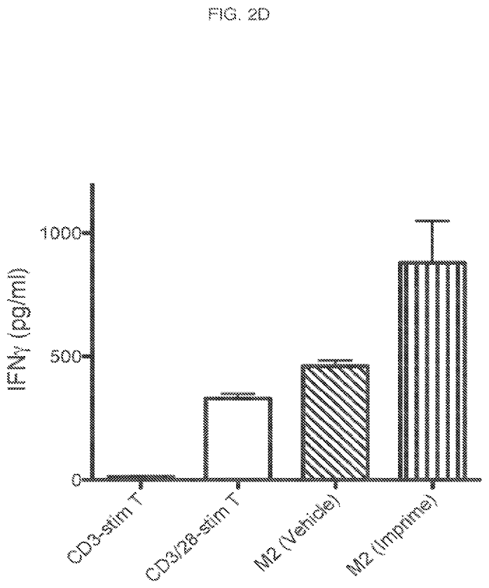

Effect of β-glucan on M2 to M1 repolarization: M1 and M2 macrophages from vehicle- or β-glucan-treated whole blood were prepared as described above. An expression of a panel of M1/M2-specific markers (including HLA-DR, CD163, CD206, CD209, CD80, CD86 and PD-L1) were measured by flow cytometry. β-glucan pretreatment did not affect M1 macrophage phenotype but did affect M2 macrophage phenotype. As shown in FIG. 2A, mean fluorescence intensity (MFI) of CD163 is downmodulated in β-glucan-treated M2 macrophages. In addition, surface expression of CD86 was enhanced as well as both protein and mRNA levels of PD-L1 (FIG. 2B).

Next, vehicle- or β-glucan-treated M1 or M2 macrophages were cultured with CD3 and CD28-stimulated, carboxy fluorscein diacetate succinimidyl ester (CFSE)-labeled autologous CD4 T cells and T cell proliferation was measured at the end of the experiment by flow cytometry, and results quantitatively reported as division index (the average number of cell divisions a population has undergone). FIG. 2C. is a representative CFSE dilution T-cell proliferation assay performed by co-culturing T-cells with β-glucan-treated M2 macrophages, and the results show the ability of the β-glucan-treated M2 macrophages to enhance CD4 T cell proliferation.

The culture supernatants from the CFSE dilution T-cell proliferation assay (FIG. 2B) were also measured for IFN-gamma levels by ELISA. FIG. 2D is a representative graph of IFN-gamma levels showing a concomitant increase in IFN-gamma production. Thus, β-glucan affects M2 to M1 repolarization and drives anti-tumorigenic Th1 polarization.

### Example 3

Effect of β-glucan on M2 to M1 repolarization in cells from high binding subjects vs. low binding subjects: Early studies evaluating binding of soluble β-glucan to neutrophils and monocytes revealed subjects have different binding capabilities. Further studies found that soluble β-glucan bound to at least some of high binding subjects immune cells, and high binding subjects also had higher levels of natural anti-β-glucan antibodies. Functional studies identified general cut-offs of binding and antibody levels, which were used identify subjects as high binders (high response to β-glucan) and low binders (low response to β-glucan).

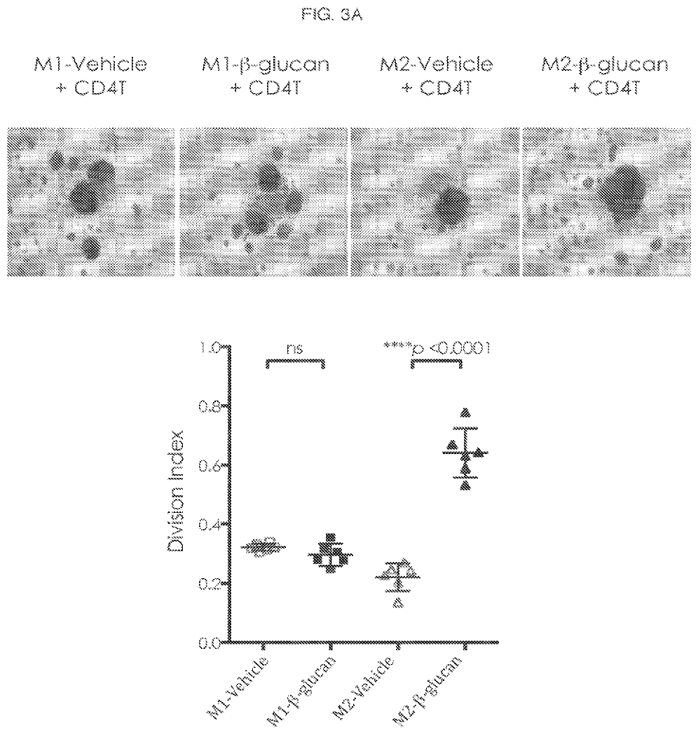

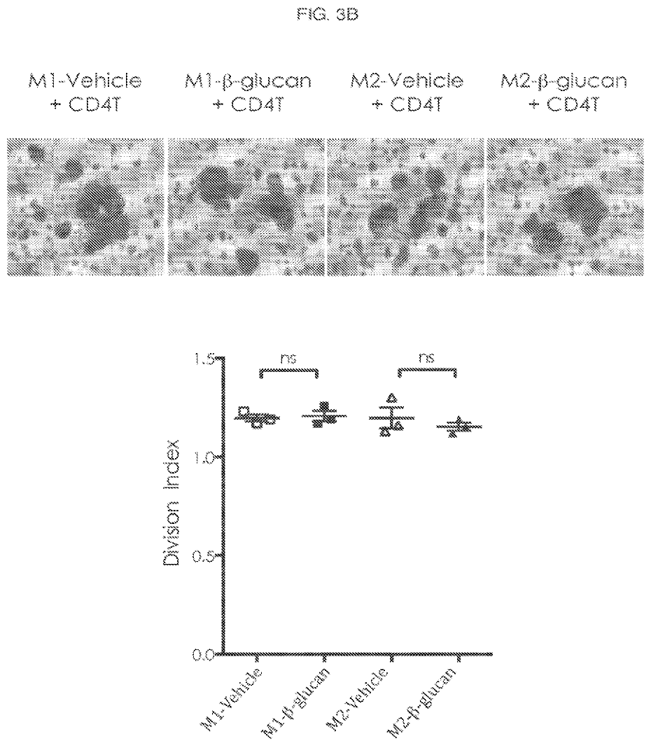

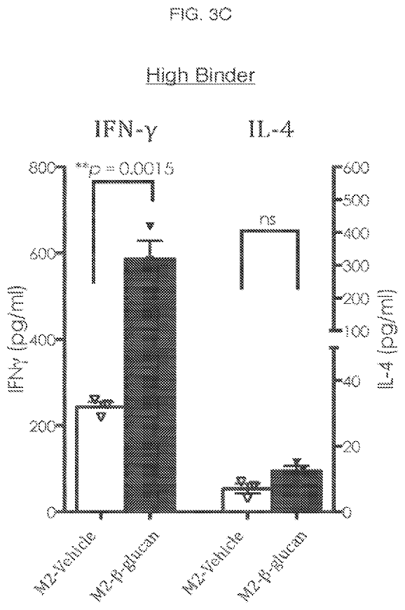

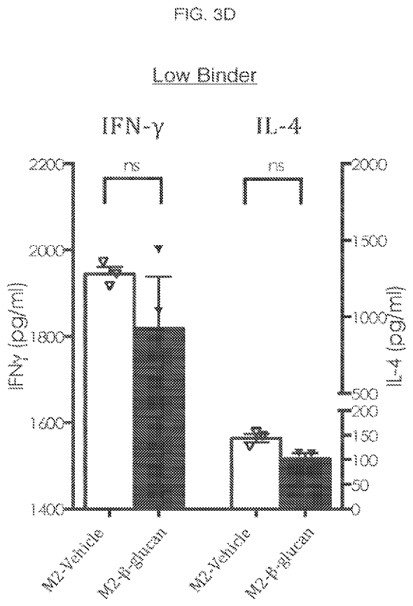

To this end, evaluations of M1/M2 macrophages derived from soluble β-glucan-treated monocytes from high binders and low binders were carried out. M1 and M2 macrophages from high binders and low binders were subsequently evaluated for A) phenotype, B) enhancement of CD4 T cell proliferation and C) modulation of IFN-γ and IL-4 production. FIG. 3A-FIG. 3C are representative results from 4 different experiments.

Expression of a panel of markers was evaluated by flow cytometry for vehicle- and p-glucan-treated, high binder-derived M1 and M2 macrophages (CD163 was evaluated twice). Median MFI was calculated for isotype control staining and surface antigen staining and the results are shown in Table 3.

TABLE 3
High Binder

| | | | CD163 | CD163 | | | | CD274 |
|---|---|---|---|---|---|---|---|---|
| | | HLA-DR | (1) | (2) | CD206 | CD209 | CD86 | (PD-L1) |
| M1 | Isotype ctrl | 58 | 58 | 103 | 84 | 98 | 139 | 58 |
| | Vehicle- | 795 | 168 | 205 | 759 | 404 | 2052 | 6130 |
| | β-glucan- | 667 | 158 | 230 | 816 | 318 | 2113 | 5563 |
| M2 | Isotype ctrl | 86 | 86 | 91 | 122 | 104 | 254 | 86 |
| | Vehicle- | 1259 | 2434 | 4079 | 1142 | 1064 | 2273 | 3087 |
| | β-glucan- | 1007 | 759 | 1153 | 1056 | 801 | 2953 | 4427 |

CD163 and CD86 were evaluated by flow cytometry for vehicle- and 11-glucan-treated, low binder-derived M1 and M2 macrophages. Median MFI was calculated for isotype control staining and surface antigen staining and the results are shown in Table 4.

TABLE 4
Low Binder

| | | CD163 | CD86 |
|---|---|---|---|
| M1 | Isotype ctrl | 58 | 278 |
| | Vehicle- | 162 | 2860 |
| | β-glucan- | 132 | 3179 |
| M2 | Isotype ctrl | 54 | 250 |
| | Vehicle- | 3500 | 2445 |
| | β-glucan- | 3315 | 2284 |

The key result is that β-glucan-treated M2 macrophages had lower expression of CD163, one of the key M2 markers. Interestingly, this result was specific for high binders as expression of CD163 in remained the same between vehicle- and β-glucan-treated M2 macrophages.

Next, the ability of M1/M2 macrophages derived from soluble β-glucan-treated monocytes from high binders and low binders to enhance CD3 & CD28-stimulated CD4 T cell proliferation was evaluated. FIG. 3A shows the results of the CD4 T cell proliferation assay in a high binder while FIG. 3B shows the results in a low binder. β-glucan-treated M2 macrophages had significantly higher ability to enhance CD3 & CD28-stimulated CD4 T cell proliferation in comparison to that observed with the vehicle-treated M2 macrophages in high binders while there was no enhanced proliferation in low binders. In addition, β-glucan-treated M1 macrophages showed no differences in this functional ability as compared to the vehicle-treated M2 macrophages in either high binders or low binders.

Modulation of IFN-γ and IL-4 production of vehicle- and β-glucan-treated M2 macrophages was then evaluated. Concomitant with enhanced proliferation, significantly increased production IFN-γ, but not IL-4 was observed in co-cultures of β-glucan-treated M2 macrophages and CD4 T cells in high binders (FIG. 3C) but not in low binders (FIG. 3D). Thus, M2 macrophages derived from β-glucan-treated monocytes are M1-like in high binder subjects.

### Example 4

Effect of β-glucan on M2 to M1 repolarization in immunosuppressive conditions: Phenotypic and functional evaluation of β-glucan-treated M2a and β-glucan-treated M2 macrophages in the presence of immunosuppressive cytokines was carried out. M2 or M2a macrophages were prepared as described above. On day 3, tumor-conditioned medium (TCM) was added M2 macrophage cultures to account for 70% of the volume of the culture and then evaluated for CD163 expression and functional activity on day 6. The TCM from BxPC3, a pancreatic cancer cell line, has been shown to contain several immunosuppressive cytokines including M-CSF, TGF-beta, IL-4, etc. M2a macrophages were cultured in IL-4 as described above.

The β-glucan-treated M2a macrophages cultured in IL-4 and M2 macrophages cultured in TCM were first evaluated for CD163 and CD86 expression. CD163 and CD86 were evaluated by flow cytometry and median MFI was calculated for isotype control staining and surface antigen staining and the results are shown in Table 5.

TABLE 5
Immunosuppressive conditions

| | | CD163 | CD86 |
|---|---|---|---|
| M2a | Isotype ctrl | 149 | 147 |
| | Vehicle- | 664 | 4130 |
| | β-glucan- | 309 | 4572 |
| M2 | Isotype ctrl | 138 | 103 |
| | Vehicle- | 1736 | 1283 |
| | β-glucan- | 1112 | 2697 |

As seen in previous M2 differentiation experiments using M-CSF, the β-glucan-treated monocytes cultured in TCM also showed marked down-regulation of CD163. In addition, the β-glucan-treated M2 macrophages had higher HLA-DR expression (data not shown).

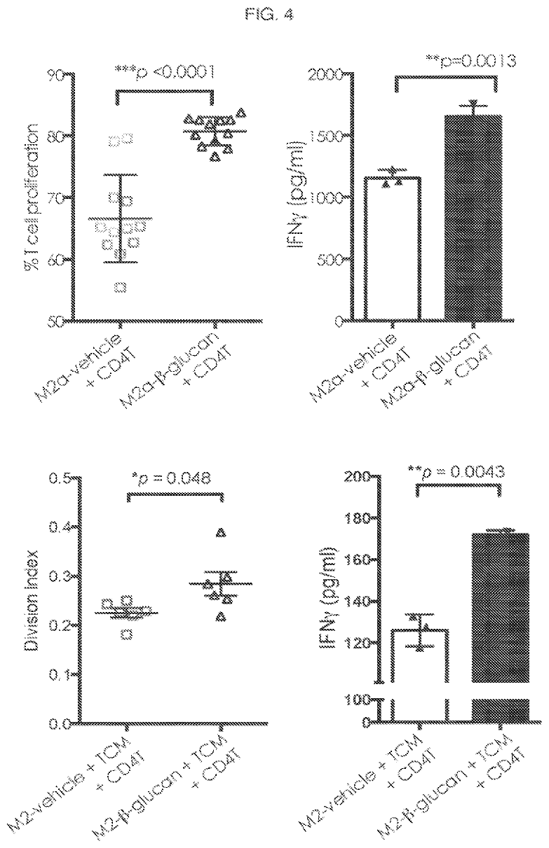

A functional evaluation of ability to modulate CD4 T cell proliferation was then performed by CD4 T cell proliferation assay (three or six replicates in each condition). β-glucan-treated M2 macrophages cultured in TCM and M2a macrophages cultured in IL-4 maintained the ability to enhance CD4 T cell proliferation in comparison to that observed with the vehicle-treated M2 and M2a macrophages. Concomitant with enhanced proliferation, significantly increased production of IFN-γ was observed in co-cultures of macrophages and CD4 T cells (FIG. 4).

The above examples demonstrate that soluble § i-glucan has the ability to inhibit M2 polarization and induce more M1-like cells as demonstrated by the reduced expression of CD163, increased expression of CD86, and by inhibiting the ability of M2 to suppress CD4 T cell proliferation. Even under immunosuppressive conditions, simulated by the presence of either IL-4 in combination with M-CSF or tumor conditioned medium (TCM), soluble β-glucan was able to inhibit M2 polarization and enhance their ability to help CD4 T cell proliferation. The enhancement of CD4 T cell proliferation by M2-soluble β-glucan was accompanied with an increase in the pro-inflammatory, Th1 polarizing cytokine, IFN-γ, and no change in the production of immunosuppressive cytokine IL-4.

### Example 5

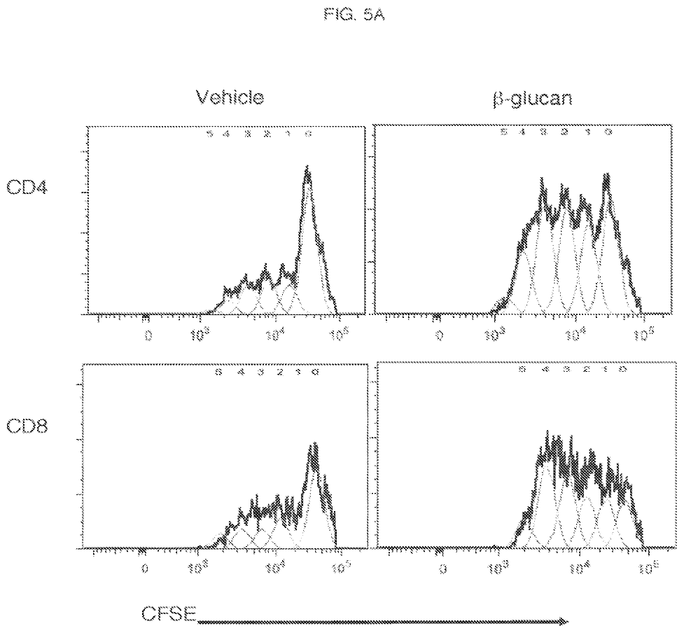

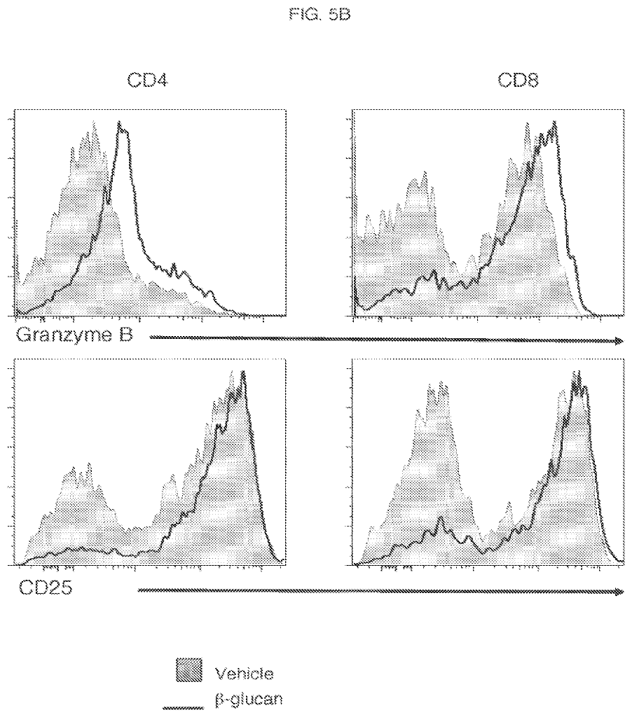

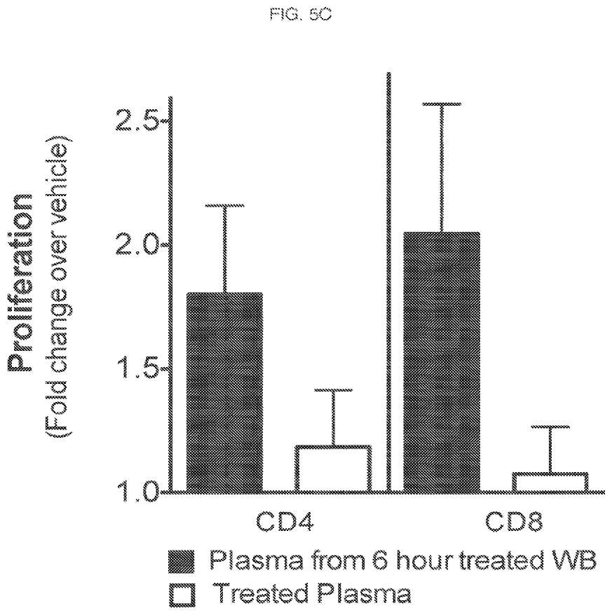

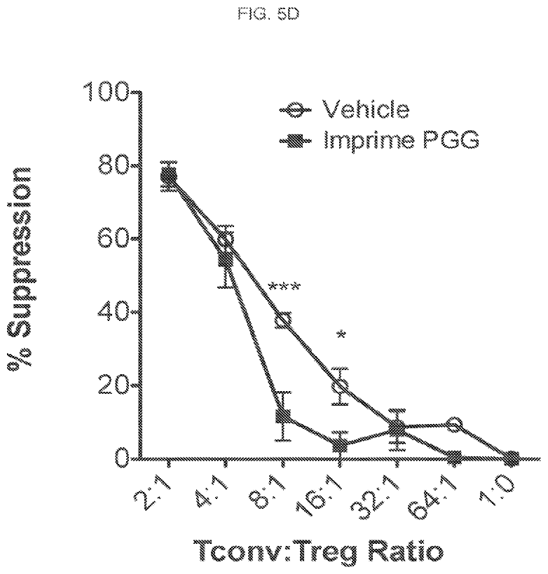

Effect of β-glucan on CD4/CD8 T cell proliferation and activation in the presence of Tregs: To obtain plasma, whole blood was treated for 6 hours with 25 μg/mL β-glucan or vehicle, spun down and the plasma removed. 50,000 autologous CFSE-labeled PBMCs were cultured in the treated plasma for 3 days in the presence of 50,000 T cell activating CD3/28 beads (DYNABEADS Human T-Activator CD3/CD28 for T Cell Expansion and Activation). At the end of the culture, PBMCs were stained with CD4 and CD8 and T cell proliferation was measured by CFSE dilution. As shown by the representative CFSE dilution plots in FIG. 5A, plasma from β-glucan-treated whole blood provided a significant enhancement in both CD4 and CD8 proliferation as compared to vehicle-treated controls.

Next, to show the effect of soluble β-glucan on CD4 and CD8 cell activation, the T cell proliferation assay described above was again carried out. At day 3, however, the cells were stained for markers of activation including Granzyme B production and CD25 upregulation. The graphs shown in FIG. 5B demonstrate that β-glucan-treated plasma enhances CD4 and CD5 cell activation.

The enhancement in proliferation was greatest when whole blood was treated for the 6 hour incubation period prior to plasma isolation, indicating that this effect on T cell proliferation is the result of an indirect mechanism (i.e. cytokine release by innate immune cells). To determine whether the enhancement of T cell proliferation by β-glucan is direct or indirect, T cell proliferation assays were carried out as described above except the plasma from untreated (vehicle) whole blood was then either treated with β-glucan or vehicle prior to adding to autologous PBMCs. CFSE dilution was quantitated by Division Index using FLOWJO software and plotted as fold change over vehicle control. The results shown in FIG. 5C indicate that β-glucan's enhanced effect is due to indirect mechanisms.

Since these PBMC cultures contain Tregs, the suppressive ability of Tregs seem to be altered in the presence of soluble β-glucan and studies were carried out to determine if β-glucan affected Treg suppression. Plasma from β-glucan-treated or vehicle-treated whole blood (described above) was added to 25,000 isolated CFSE-labeled autologous CD4 T cells (CD4⁺CD25⁺) along with increasing numbers of isolated autologous Tregs (CD4′CD25*) resulting in wells with increasing ratios. Cells were then stimulated with 50,000 T cell activating CD3/28 beads for 3 days. Proliferation was subsequently measured by CFSE dilution and quantified by Division Index, which was used to calculate the % suppression of Tregs in the co-culture. % suppression=100-(Division Index of Treg well/Division Index of 1:0 well)/100. The results are shown in FIG. 5D. Plasma from β-glucan-treated whole blood showed significant decreases in the suppressive capacity of Tregs as compared to plasma from vehicle-treated whole blood.

Treg suppression by β-glucan also resulted in enhanced IFN-gamma production. The Treg suppression assay was conducted as described above, and after 3 days of co-culture, supernatants were analyzed for IFN-gamma production. FIG. 5E shows the results of the IFN-gamma production from wells cultured at an 8.1 T cell to Treg ratio. Taken together, these results show that β-glucan affects CD4 and CD8 proliferation along with Treg function resulting in enhanced anti-tumor adaptive effector function.

### Example 6

Establishment and characterization or in vitro cultured human immature monocyte-derived dendritic cells (imMoDC) and mature monocyte-derived dendritic cells (mMoDC). Given that macrophages and dendritic cells are the two key antigen presenting cell types that bridge innate and adaptive immunity, the phenotypic and functional effect of soluble β-glucan was also evaluated on human monocyte-derived dendritic cells (MoDC). Monocytes enriched from soluble β-glucan- or vehicle-treated whole blood were cultured in media containing the appropriate cytokines, GM-CSF plus IL-4, for differentiation of dendritic cells Steps included in the method for in vitro culture and characterization of human MoDCs are outlined below.

Whole blood (Untreated or treated with soluble β-glucan or vehicle
for 2 hours at 37° C.)

[symbol]

Isolate PBMC by Ficoll separation

[symbol]

Enrich monocytes by negative selection

[symbol]

Culture monocytes in XVivo15 medium (supplemented with 10% human
autologous serum, 50 ng/ml rhGM-CSF and 50 ng/ml rhIL-4) for 6-7 days

[symbol]

Induce maturation of MoDC by LPS (50 ng/ml) + TNF-α (25 ng/ml)
for 48-72 hours

[symbol]

Morphology of MoDC determined by microscopy
Phenotypic analysis by flow cytometry
Functional study by allogeneic mixed lymphocyte reaction (allo-MLR)

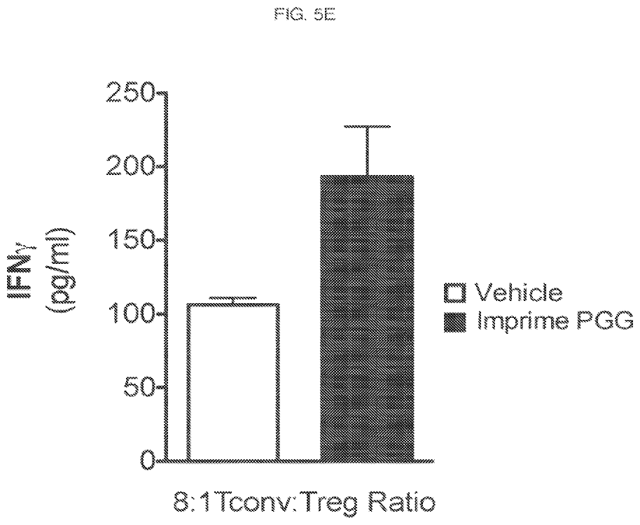

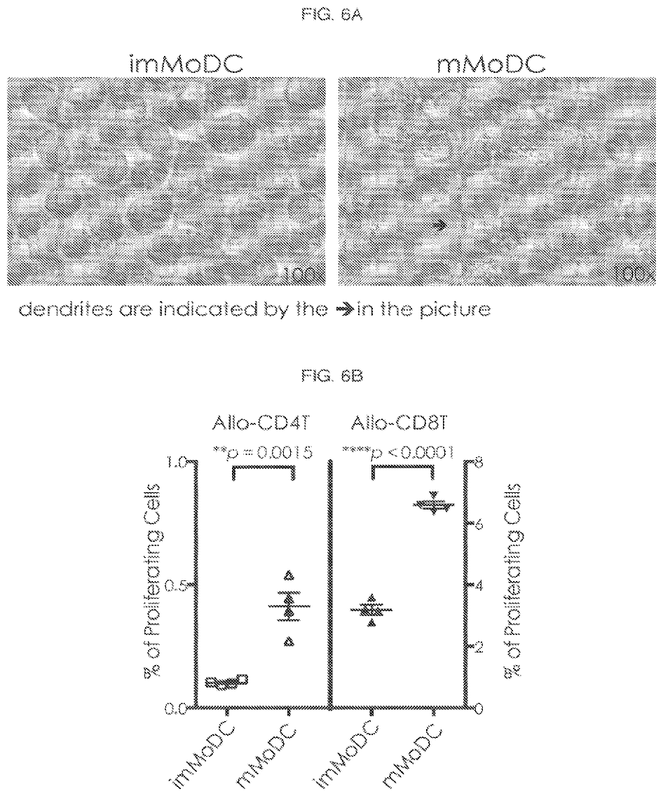

imMoDCs and mMoDCs, prepared as described above, are shown in FIG. 6A. The morphology of mMoDCs is characterized by the presence of long projections or dendrites.

mMoDCs were evaluated for CD80, CD83, CD86 and HLA-DR expression by flow cytometry, and median MFI was calculated for isotype control staining and surface antigen staining and the results are shown in Table 6.

TABLE 6

| | | CD80 | CD86 | CD83 | HLA-DR |
|---|---|---|---|---|---|
| MoDC | Isotype ctrl | 143 | 93 | 120 | 21 |
| | imMoDC | 165 | 1554 | 134 | 132 |
| | mMoDC | 492 | 35637 | 448 | 470 |

mMoDC showed increased surface expression of the maturation and co-stimulatory markers CD80, CD83, CD86 as well as HLA-DR. Furthermore, these mMoDC also showed immunogenicity in an allogeneic mixed lymphocyte reaction (four replicates in each condition), triggering increased CD4 and CD8 T cell expansion (FIG. 6B).

### Example 7

Effect of β-glucan on maturation of MoDCs: The phenotypic and functional evaluation of mMoDC prepared from soluble β-glucan-treated whole blood of a high binder and low binder was carried out. mMoDCs from a high binder and a low binder were prepared as described above. mMoDCs were evaluated for CD80, CD83, CD86 and HILA-DR expression by flow cytometry, and median MF1 was calculated for isotype control staining and surface antigen staining and the results are shown in Table 7.

TABLE 7
mMoDCs

| | | CD80 | CD86 | CD83 | HLA-DR |
|---|---|---|---|---|---|
| High | Isotype ctrl | 223 | 151 | 162 | 151 |
| binder | Vehicle- | 640 | 871 | 168 | 4286 |
| | β-glucan- | 744 | 10759 | 466 | 7049 |
| Low | Isotype ctrl | 213 | 141 | 162 | 30 |
| binder | Vehicle- | 1056 | 67664 | 2346 | 3561 |
| | β-glucan- | 1197 | 77749 | 2924 | 2960 |

The increased expression of CD80, CD86, CD83 and HLA-DR on β-glucan-treated mMoDC derived from high binders indicate that these mMoDCs are more mature than those derived from low binders.

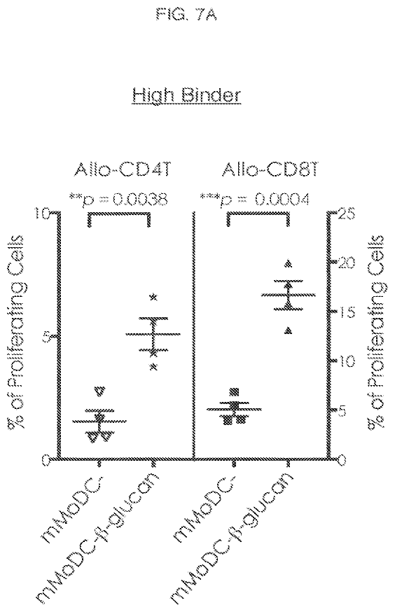

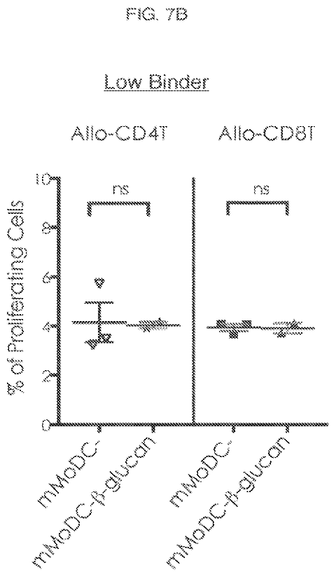

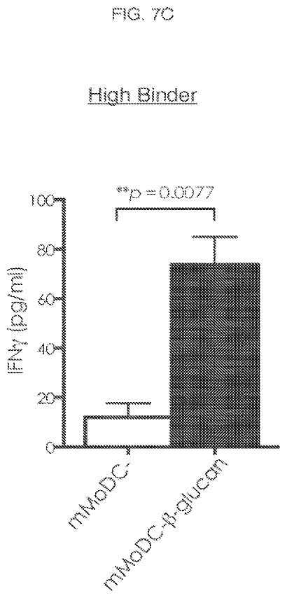

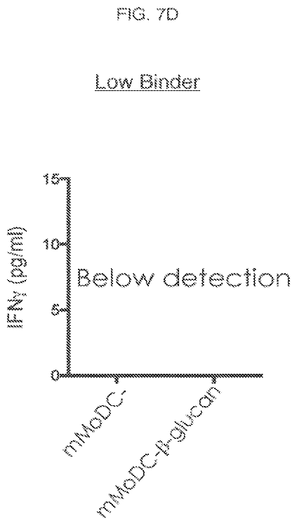

The β-glucan-treated m MoDC derived from high binders also showed increased immunogenicity in an allo-MLR (four replicates in each condition), again triggering increased CD4 and CD8 T cell expansion (FIG. 7A) over cells derived from low binders (FIG. 7B).

In addition, the β-glucan-treated mMoDC derived from high binders was able to modulate IFN-γ production over cells derived from low binders and vehicle-treated mMoDCs (FIG. 7C).

MoDC derived from β-glucan-treated monocytes are more mature even in immunosuppressive conditions. MoDCs were prepared as described above. TCM was added to account for 70% of the volume of the culture on day 0 and was present throughout the culturing period. mMoDCs cultured in the presence of TCM were subsequently evaluated for phenotypic changes. Median MF1 was calculated for isotype control staining and surface antigen staining and results are shown in Table 8.

TABLE 8
Immunosuppresive Conditions

| | | CD80 | CD86 | CD83 | HLA-DR |
|---|---|---|---|---|---|
| MoDC | Isotype ctrl | 120 | 88 | 118 | 31 |
| | Vehicle- | 439 | 3340 | 274 | 352 |
| | β-glucan- | 465 | 15797 | 607 | 361 |

### Example 8

Cell-to-cell contact and soluble factors increase CD4 T cell proliferation by S-glucan-treated M2 macrophages: Using CD4 T cell proliferation as the read-out, the requirement of cell-to-cell contact or soluble factor(s) in initiating the proliferation by β-glucan-treated M2 macrophages was studied. To study cell-to-cell contact between macrophages and T cells, CD4 T cell proliferation was measured when co-cultured with β-glucan-treated M2 macrophages in the absence of CD28 co-stimulation and modulation of surface activation markers was studied on both β-glucan-treated M2 macrophages and T cells in the co-culture.

Evaluation of cell-to-cell contact was carried out as follows: vehicle- and β-glucan-treated M2 macrophages and CD3 & CD28− versus CD3 only-stimulated CD4 T cell co-cultures were utilized for measuring CD4 T cell proliferation and IFN-γ production.

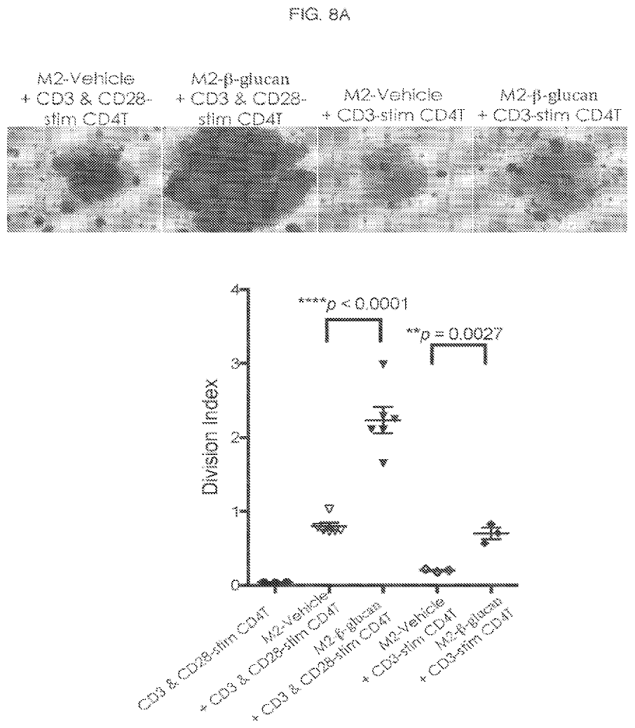

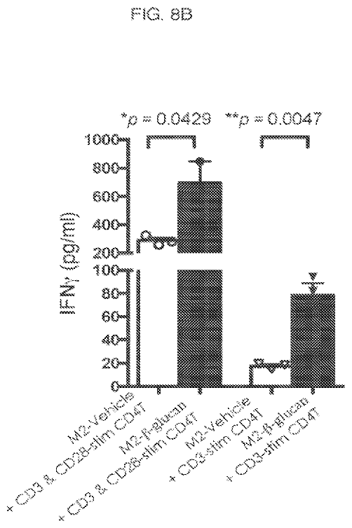

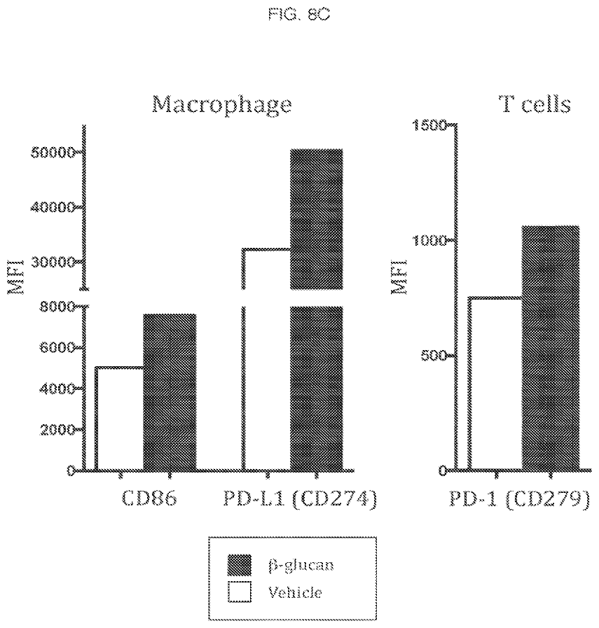

As shown in HG 8A, β-glucan-treated M2 macrophages cultured with CD4 T cells in the absence of exogenous CD28 antibody demonstrated significantly higher ability to enhance CD4 T cell proliferation. Concomitant with enhanced proliferation, significantly increased production of IFN-γ was observed in co-cultures of β-glucan-treated M2 macrophages and CD4 T cells (FIG. 8B).

Changes in surface marker expression on both macrophages and CD3 & CD28-stimulated T cells were also measured. Vehicle- and β-glucan-treated M2 macrophages and CD T cells from the co-cultures were evaluated by flow cytometry for the modulation of co-stimulatory or co-inhibitory molecules. FIG. 8C and Table 9 are representative results from 2 different experiments.

TABLE 9

| | Change in MFI on | Change in MFI |
|---|---|---|
| Surface Markers | Macrophages* | on T cells* |
| HLA-DR | No change | NA |
| CD86 | Increase | NA |
| CD80 | No change | NA |
| CD28 | NA | No change |
| CTLA-4 | NA | No change |
| CD40 | No change | NA |
| CD40L | NA | No change |
| 4-1BBL | No change | NA |
| 4-1BB | NA | No change |
| OX40 | No change | NA |
| PD1 (CD279) | NA | Increase |
| PD-L1 (CD274) | Increase | NA |
| CD209 | No change | NA |
| CD172 | No change | NA |

*Change in MFI on β-glucan-treated M2 macrophages/T cells relative to that observed in vehicle-treated M2 macrophages/T cells

Of all the surface markers tested, a relative increase in surface expression of CD86 (day 8) and PD-L (day 9) was observed on the surface of β-glucan-treated M2 macrophages as compared to that on the vehicle-treated M2 macrophages. Increased expression of PD-1 (day 9) was observed on the surface of T cells co-cultured with β-glucan-treated M2 macrophages.

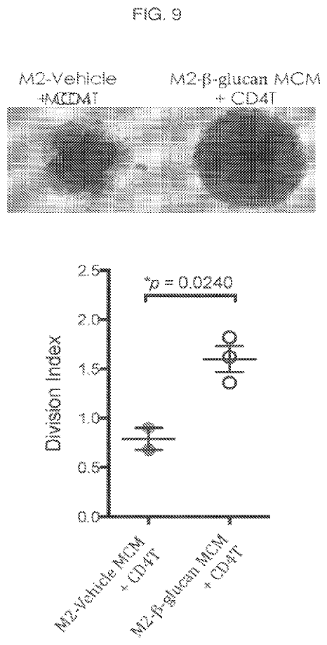

To determine whether soluble factors secreted from β-glucan-treated M2 macrophages are required, measurements were carried out of CD4 T cell proliferation co-cultured with β-glucan-treated M2 macrophages MCM (50% of volume) and surface activation marker modulation on T cells incubated with the MCM were observed. FIG. 9 is representative of 2 different experiments. When evaluated by CD4 T cell proliferation assay, β-glucan-treated M2 macrophage MCM cultured with CD4 T cells demonstrated significantly higher ability to enhance CD4 T cell proliferation (FIG. 9) in comparison to the vehicle-treated M2 macrophage MCM. In addition, the CD4 T cells cultured in vehicle- and β-glucan-treated M2 macrophage MCM were evaluated for modulation of co-stimulatory or co-inhibitory molecules (CD80, CD28, CTLA-4, 4-13BB and PD-1). Surprisingly, no change in any of the T cell markers was observed (data not shown).

### Example 9

Analysis of β-glucan-treated M2 macrophages in high binders vs. low binders: As discussed previously, anti-β-glucan antibody (ABA) thresholds in subjects have been shown to be important for β-glucan immunotherapy. Therefore, the importance of ABA threshold in β-glucan's ability to modulate M1/M2 polarization was investigated. β-glucan's ability to modulate M1/M2 polarization in high binders versus low binders was determined by both phenotypic and functional evaluations.

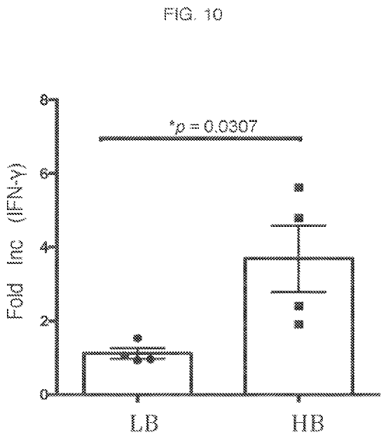

M2 macrophages from 4 high binders and 4 low binders were prepared and evaluated for their ability to modulate CD4 T cell proliferation. The supernatants from the various CD4 T cell proliferation conditions were measured for IFN-γ by ELISA. Results shown in FIG. 10 are representative from 4 different experiments. Fold change over the IFN-7 levels produced in the co-cultures of vehicle-treated M2 macrophages and CD4 T cells are plotted for each of the 4 donors.

In low binders, β-glucan did not modulate any of the phenotypic markers on the monocyte-derived macrophages in M1/M2 polarizing conditions (data not shown), and in a functional evaluation of low binders by CD4 T cell proliferation assay, the β-glucan-treated M2 macrophages neither enhanced CD4 T cell proliferation nor increased IFN-γ production.

### Example 10

Serum cross-over studies: Because β-glucan failed to show modulation of M1/M2 polarization in low binders, modulation by β-glucan using monocytes from a low binder in the presence of serum containing higher levels of ABA (serum cross-over from a high binder) was evaluated. To test this, M2 macrophages were prepared as described above with a few modifications. The whole blood of a low binder was spun down to remove the plasma and then the cells were reconstituted with serum obtained from a high binder. The reconstituted blood was treated with vehicle or β-glucan (25 μg/mL) for 2 hours at 37° C. The monocytes were evaluated for binding by using anti-β-glucan specific monoclonal antibody and subsequent flow cytometry. The vehicle or β-glucan-treated monocytes in whole blood were then isolated and differentiated to M2 macrophages, and either the M2 cells (data not shown) or the MCM were used to evaluate for their ability to enhance CD4 T cell proliferation (six replicates in each condition) and increase IFN-γ production using methods described above.

Monocytes in the whole blood of a low binder did not bind β-glucan but showed significantly higher binding when a high binder's serum containing higher levels of ABA was added to the low binder's whole blood (FIG. 11A) In addition, MCM from β-glucan-treated M2 macrophages of a low binder crossed-over with a high binder's serum have significantly higher ability to enhance CD4 T cell proliferation in comparison to that observed with the vehicle-treated M2 macrophages. Concomitant with enhanced proliferation, significantly increased production of IFN-γ was observed in co-cultures of β-glucan-treated M2 macrophage and CD4 T cells (FIG. 11B).

### Example 11

PD-L1 upregulation on @-glucan-treated M2 macrophages cultured in the presence of immunosuppressive cytokines (TCM): Monocytes or M2 macrophages were prepared as described above. On day 3, TCM was added to account for 70% of the volume of the culture and then evaluated for PD-L1 expression with TCM and then again when co-cultured with CD4 T cells.

β-glucan-treated M2 macrophages cultured in the TCM had higher surface expression of PD-L1 (FIG. 12A). There was also increased expression when co-cultured with CD4 T cells (FIG. 12B).

Using the above system, it was determined that β-glucan has the ability to inhibit M2 polarization as demonstrated by the reduced expression of a key M2 marker, CD163, and by inhibiting the ability of M2 to suppress CD4 T cell proliferation. Even under an immunosuppressive environment, stimulated by the presence of either IL-4 in combination with M-CSF (data not shown) or tumor-conditioned medium (TCM), β-glucan was able to inhibit M2 polarization and enhance their ability to help CD4 T cell proliferation. The enhancement of CD4 T cell proliferation by M2-β-glucan was accompanied with an increase in the pro-inflammatory, Th1 polarizing cytokine, IFN-γ, and no change in the production of immunosuppressive cytokine IL-4. As expected with increased T cell activation and IFN-γ production, increases in surface expression of PD-L1 on β-glucan-treated M2 macrophages and PD-1 on T cells were observed. The β-glucan-treated M2 macrophages themselves, as well as the soluble factors secreted by the cells are important for enhancing CD4 T cell proliferation. Lastly, β-glucan inhibited M2 polarization in only the cells from healthy donors having higher levels of ABA.

### Example 12

PD-L1 upregulation in MiaPaCa: β-glucan- and vehicle-treated M2 macrophages and β-glucan-treated M2 macrophages+ABA were cultured with high binder serum and low binder serum to evaluate PD-L1 expression on tumor cells. FIG. 13 shows that β-glucan-treated M2 macrophages increased expression of PD-L1 on tumor cells in high binders, and with addition of ABA, β-glucan-treated M2 macrophages also increased expression of PD-L1 on tumor cells in low binders.

### Example 13

Effect of soluble β-glucan on myeloid-derived suppressor cells (MDSC): MDSC accumulate in the blood, lymph nodes, and bone marrow and at tumor sites in most patients and experimental animals with cancer and inhibit both adaptive and innate immunity MDSC are induced by tumor-secreted and host-secreted factors, many of which are pro-inflammatory molecules. The induction of MDSC by proinflammatory mediators led to the hypothesis that inflammation promotes the accumulation of MDSC that down-regulate immune surveillance and antitumor immunity, thereby facilitating tumor growth.

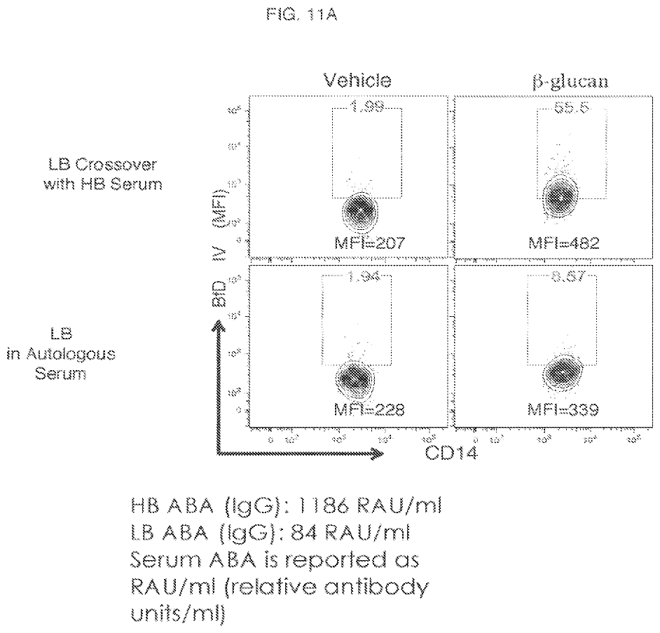

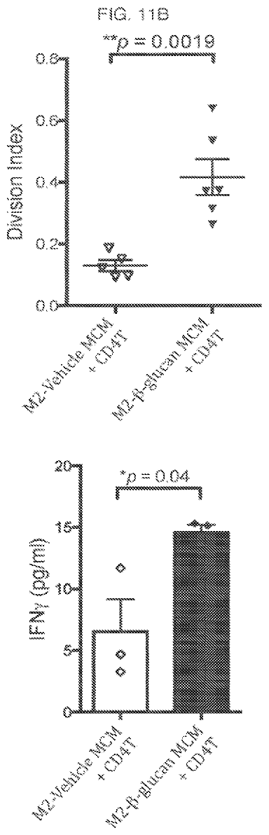

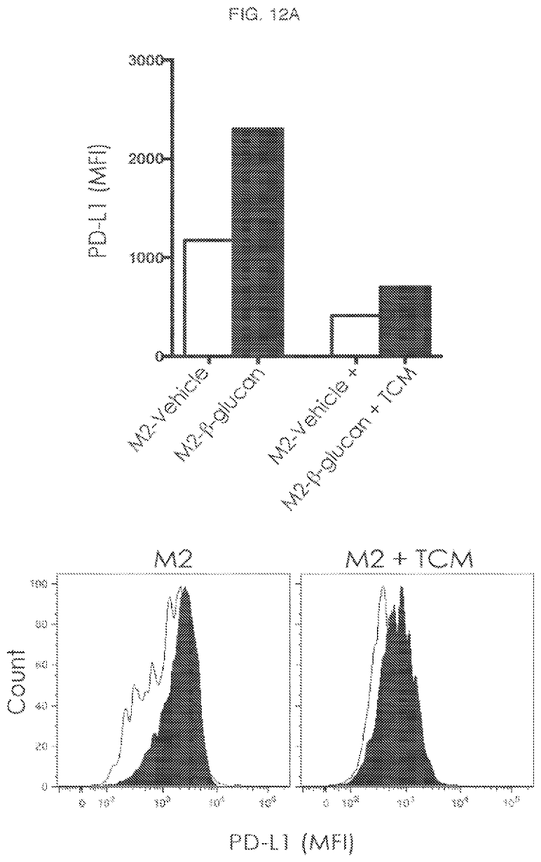

Blood was drawn at various times from a case study subject undergoing treatment with IMPRIME PGG and analyzed for the presence of MDSC. The first blood draw was done pre-infusion, cycle 8, day 1. As shown in FIG. 14A, a large population of CD33′, MDSC are present in peripheral blood. A second blood draw was done post-infusion, cycle 8, day 1. As shown in the second panel of FIG. 14A, within hours post-infusion the MDSC transiently disappear. The last blood sample was drawn pre-infusion, cycle 8, day 15. The CD33′ MDSC are again present in the peripheral blood.

In another study, human cord blood was enriched for CD34⁺ cells and cultured for 9 days to produce CD33⁺CD1b⁺ cells (MDSC). The MDSC were then treated with soluble β-glucan or citrate buffer (control) and evaluated for their ability to suppress T cell proliferation. The T cell proliferation assay was carried out at a 2:1 ratio of CD8 T cells to treated or untreated MDSCs. As shown in FIG. 14B, β-glucan-treated MDSC were less suppressive to T cell proliferation.

These results indicate that β-glucan modulates the MDSC population making them transiently leave the peripheral blood circulation and less suppressive to T cell proliferation. Thus, if one or more cancer immunotherapeutic drugs or chemotherapeutic drugs are administered in combination with soluble β-glucan, especially during the period of transiently loss of the CD33⁺ cell population, the therapies would be more effective against the tumors.

### Example 14

Supernatant from β-glucan-treated M2 macrophages/MoDC and T cell co-culture induces PD-L1 expression on tumor cells: M2 macrophages and MoDC were prepared as described above. The macrophages and the MoDC were subsequently used in T cell proliferation assays as described previously. The supernatants from these proliferation assays were harvested and incubated with various tumor cell lines, including NSCLC, breast, pancreatic, colon, and B cell lymphoma. The expression of PD-L1 on these tumor cell lines were evaluated post 48 hours by flow cytometry. Shown in FIG. 15 are representative results from 3 different experiments.

T cells require three signals for their effector mechanisms. Signal 1 is the antigen presented in the context of MIC molecules on the antigen presenting cells (APC), signal 2 is provided by the membrane costimulatory molecules on the APC, and signal 3 is the cytokines produced in the milieu for effector function. Coinhibitory molecules, such as PD-L1 can inhibit the effector functions of T cells.

Macrophages and dendritic cells derived from β-glucan-treated monocytes in vitro have higher expression levels of PD-L1, but the treatment also increases the expression of the costimulatory molecule CD86, (signal 2), and cytokines (signal 3) allowing for enhanced T cell effector function.

The broader, innate and adaptive immune response elicited by β-glucan also enhances PD-L expression on the tumor cell lines. These results demonstrate that the up regulation of PD-L1 expression induced by β-glucan on both the immune and the tumor cells makes it a promising combination partner with the checkpoint inhibitor cancer immunotherapy.

It is equally important to note that β-glucan also has the capability to offset the inhibitory effect of PD-L1 up regulation by compensatory mechanisms such as increased expression of costimulatory molecules and production of immunostimulatory cytokines.

### Example 15

Effect of soluble β-glucan in combination with anti-angiogenic agents on the TME: Tumor angiogenesis alters immune function in the TME resulting in an immunosuppressive environment. Anti-angiogenic agents, such as anti-VEGFR2 antibody DC101 (mouse ramucirumab), have proven useful in cancer therapy. Because soluble β-glucan can skew the TME to a more anti-tumor environment, it was used in combination with DC101 to treat NCI-H441 non-small cell lung cancer (NSCLC) subcutaneous xenografts in mice to increase the effectiveness of the DC101 antibody.

6 to 8 week-old female athymic nude mice were injected with 5×10⁶ H441 tumor cells in a volume of 0.2 ml subcutaneously in the flank. Mice were dosed biweekly when the mean tumor volume reached about 150 mm³ with the following agents:
0.2 ml/mouse vehicle
1.2 mg/mouse IMPRIME PGG (Biothera, Inc.)
10 mg/kg or 20 mg/kg DC101 (Clone: DC101 Catalog #: BE0060)
   Blood samples were collected on day 10 and 2 hours after the last dose.

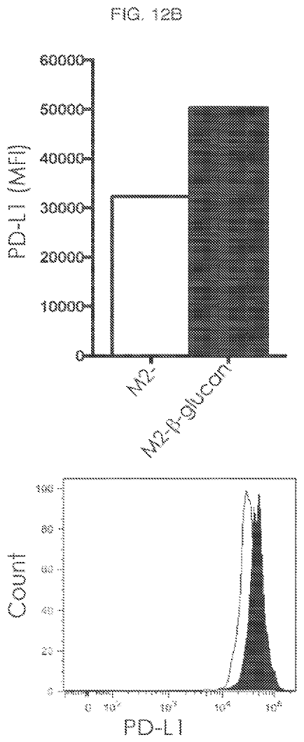

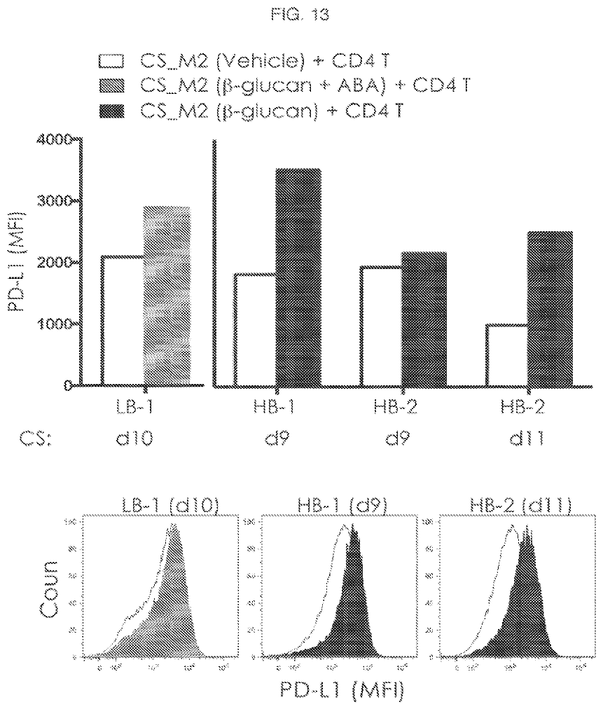

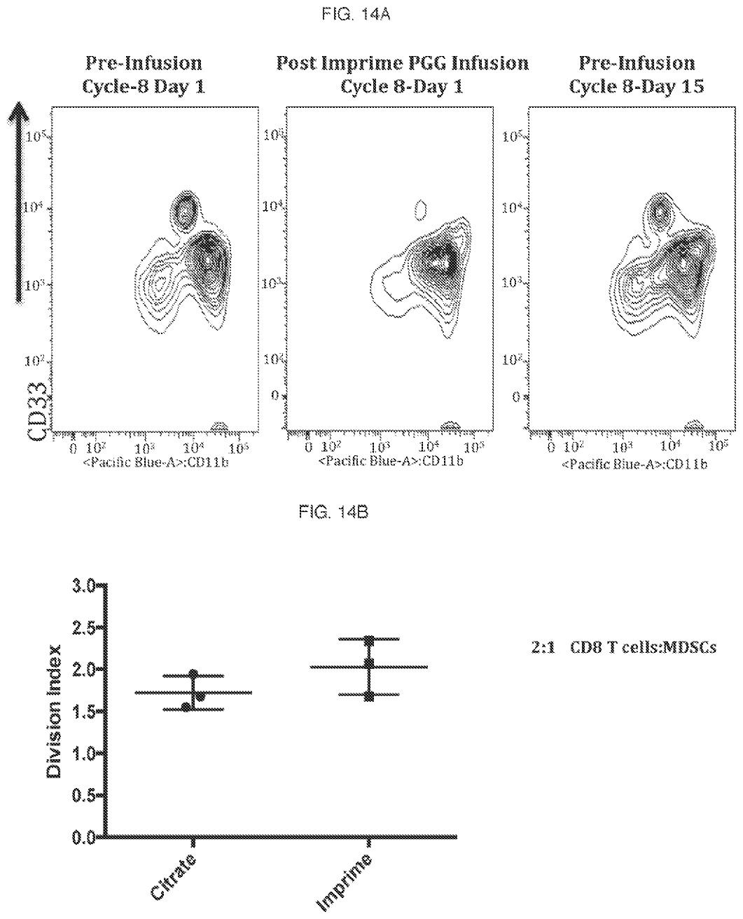

Treatment groups included vehicle (PBS control), IMPRIME PGG alone, DC10⁵ alone and DC101+IMPRIME PGG Tumors were randomized to treatment groups once sizes reached a group mean of 150 mm³. The results of tumor volumes from the 10 mg/kg treatment groups are shown in FIG. 16A and FIG. 16B.

As is evident from the graphs, IMPRIME PGG+DC101 (a complement-activating, non-tumor targeting antibody) acted synergistically to minimize growth of the tumor. As specifically shown in FIG. 16B, 70% of mice treated with IMPRIME PGG+DC10⁵ had greater than 75% tumor growth inhibition (TG) compared to just 30% of mice treated with DC101 alone. Thus, soluble β-glucan in combination with an anti-angiogenic agent (which may or may not be a complement-activating, non-tumor targeting antibody) is an effective cancer therapy.

Additional analysis was performed to show that immune cells are activated in the TME. At day 37, post injection, spleens were harvested and single cell suspensions were harvested by FACS. Frequency or GMFI was calculated in FLOWJO after gating for cells indicated in FIG. 16C. As shown in the upper graphs of FIG. 16C, animals treated with IMPRIME PGG+DC101 have reduced splenic MDSCs while the lower graphs show an increase in splenic macrophages in the combination treated animals.

Additional analysis was also carried out on whole tumors removed from the mice at day 37, post injection. Single cell suspensions from 1441 tumors were generated according to protocol utilizing a Milteny Octodissociator. Total RNA was isolated from tumor suspensions with a RNeasy minikit (QIAGEN, Venlo, Limburg). First-strand cDNA was synthesized with SuperScript H First-Strand Synthesis SuperMix (Life Technologies, Carlsbad, Calif.). Real-time quantitative polymerase chain reaction (qRT-PCR) was performed using TaqMan Gene Expression Master Mix and Taqman Gene Expression assay mix containing sequence-specific primers for TNFα, CD206, TGFβ and 18s rRNA (Life Technologies) with a Step One Plus Real-Time PCR System (Life Technologies). Data quantitation was done using the comparative Δ-ΔCt method. The amount of mRNA in each sample was normalized using the mean 18s rRNA levels. The fold change of TNF-α, CD206, and TGF-β in the IMPRIME PGG+DC101 treated group was compared with that of the vehicle control and DC101 alone group. As shown in FIG. 16D, expression of TNF-α increased while expression of CD206 and TGF-β decreased in animals treated with IMPRIME PGG+DC101, which indicates an increase in cells with a Th1-like phenotype within the TME.

### Example 16

Soluble β-glucan in combination with anti-PD-L1 antibodies enhances tumor-free survival: in another animal study, mice were injected with MC38 tumor cells and randomized into treatment groups. 8 to 12 week-old female C57BL/6 mice were injected with 1×10⁶ MC38 tumor cells, a colon adenocarcinoma that expresses low levels of PD-L1, in a volume of 0.1 ml injected subcutaneously in the flank. Mice were dosed biweekly starting on day 3 with the following agents:
0.2 ml/mouse vehicle
1.2 mg/mouse IMPRIME PGG (Biothera, Inc.)
100 μg/mouse anti-PD-L1 Clone: 10F.9G2 BioXcell Catalog #: BE0101
   Blood samples were collected 1 hour prior to dose 1, 2 hours after dose 3, the endpoint and 2 hours after the last dose (day 20). Treatment groups included vehicle (PBS control), IMPRIME PGG alone, anti-PD-L1 antibody alone and anti-PD-L1+IMPRIME PGG. Tumors were randomized to treatment groups once sizes reached a group mean of 150 mm³. The results are shown in Table 10.

TABLE 10

| Treatment Groups | Tumor-free Survivors (day 29) |
|---|---|
| Vehicle | 1/18 |
| IMPRIME PGG | 2/18 |
| Anti-PD-L1 | 6/18 |
| Anti-PD-L1 + IMPRIME PGG | 14/17 |

To assess the durability of this response, these mice were re-challenged by injection of MC-38 tumor cells on the opposite flank. These mice remained tumor-free even while MC-38 tumors readily grew in age-matched, tumor-naïve, control mice. Again, the combination of anti-PD-L1 antibody+soluble β-glucan worked synergistically to effectively enhance tumor-free survival and foster immunologic memory.

It should also be noted that PD-L1 expression on tumors is a biomarker for anti-PD-1 antibody responsiveness. Therefore, because soluble β-glucan induces PD-L1 expression on tumors, soluble Ji-glucan will also enhance the effectiveness of anti-PD-1 antibodies. Th1s is confirmed by the increased PD-1 expression induced by soluble β-glucan treatment described above.

### Example 17

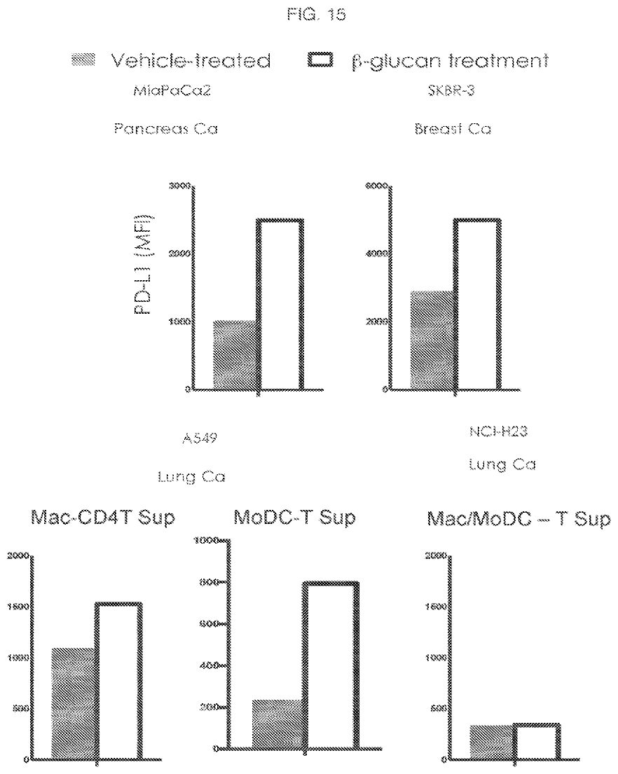

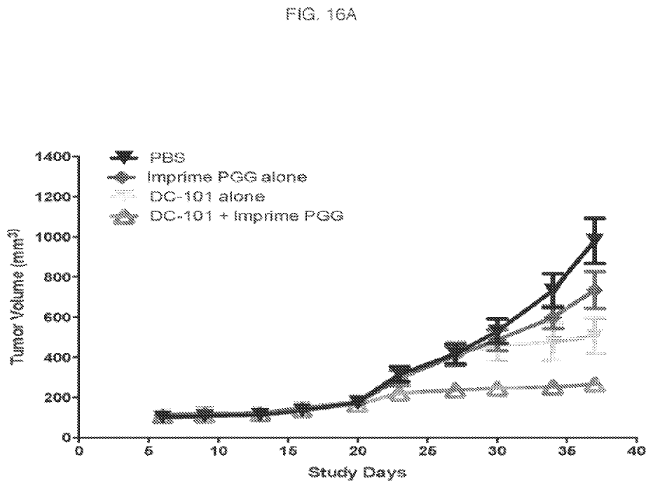

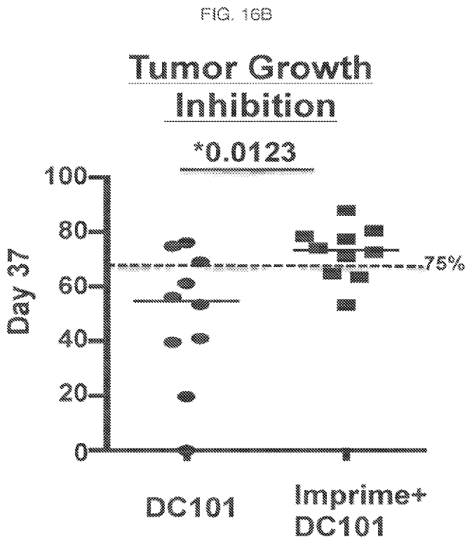

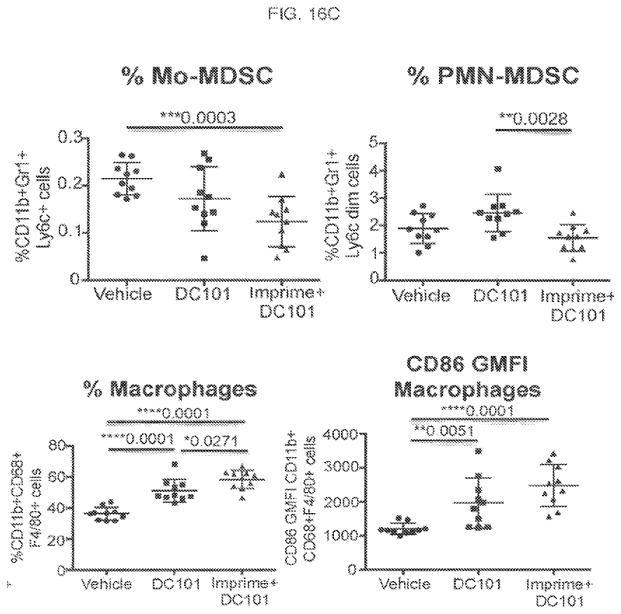

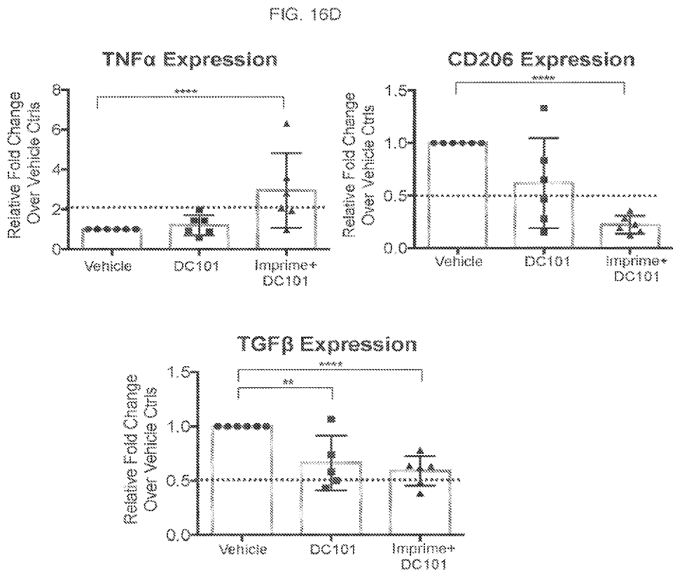

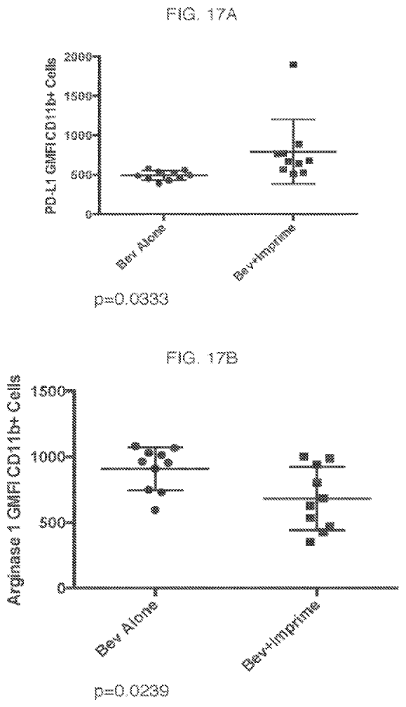

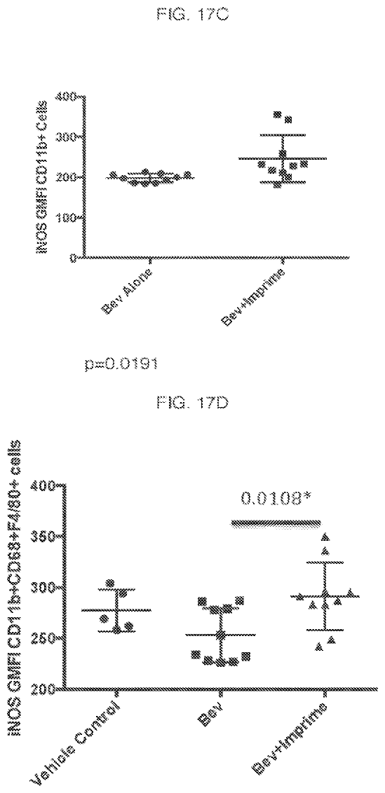

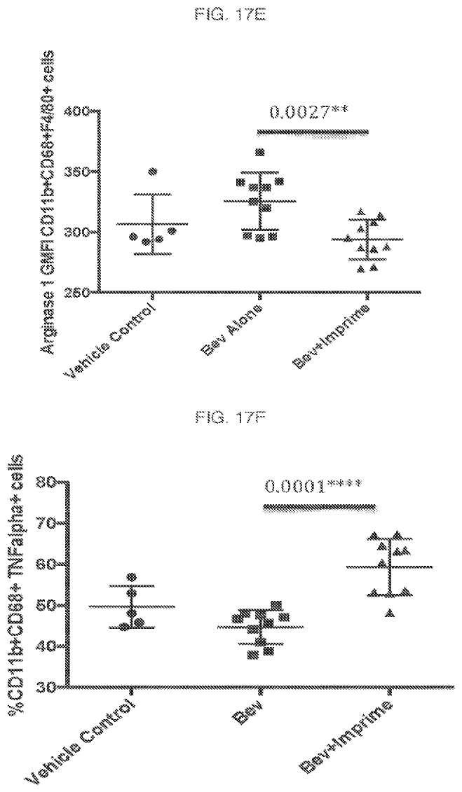

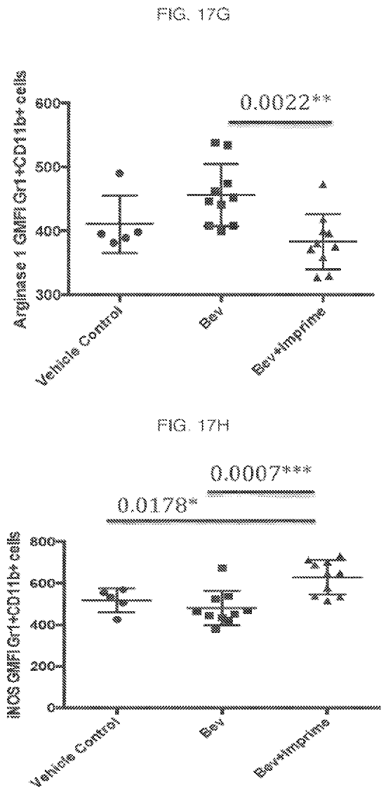

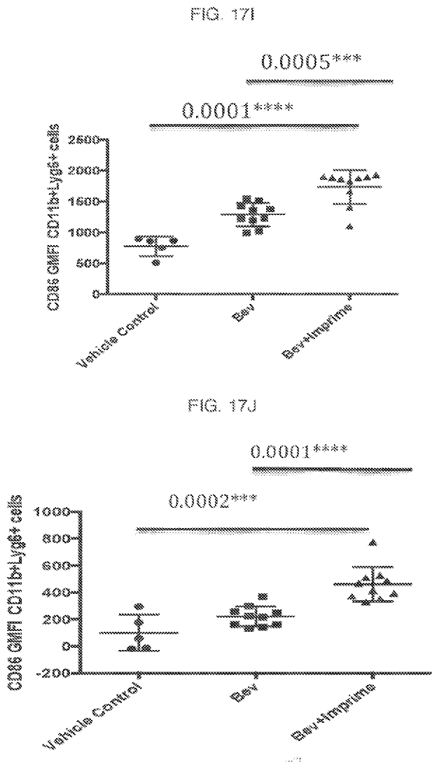

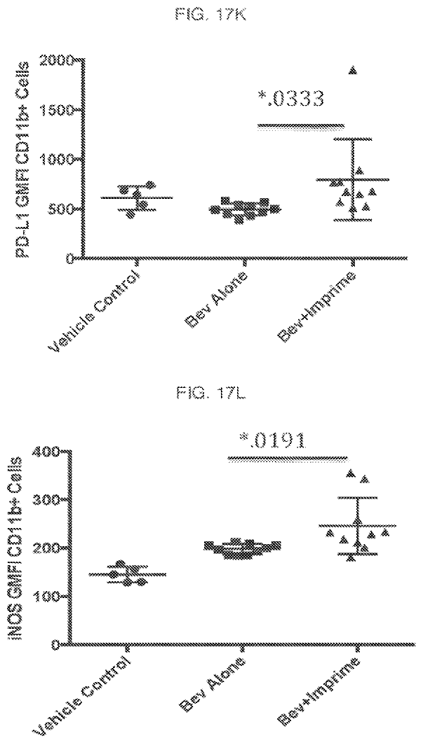

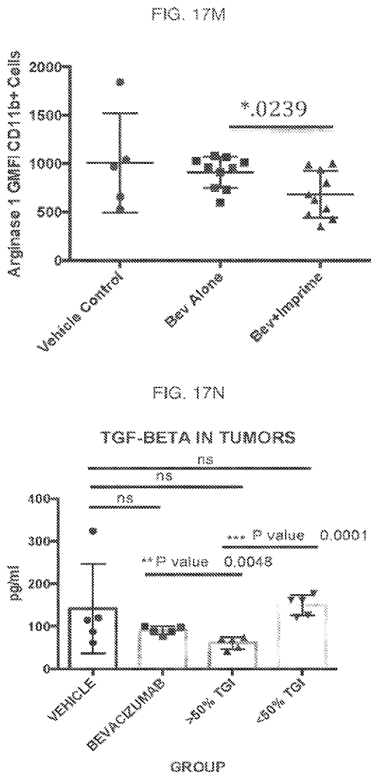

In vivo effect on TME of soluble β-glucan and anti-angiogenic agents: Mice bearing H1299 NSCLC tumors were administered bevacizumab (an anti-angiogenic antibody) (5 mg/kg twice weekly IP for four weeks) alone or in combination with IMPRIME PGG (1.2 mg/mouse twice weekly IV for four weeks) as described above for the other mouse studies. As shown in FIG. 17A, the treatment group administered the combination of bevacizumab and IMPRIME PGG showed an increase in PD-L1 expression, FIG. 17B shows down-modulation of Arginase 1 and FIG. 17C shows an increase in inducible nitric oxide synthase (iNOS) expression in the C11b positive innate immune infiltrate of the TME as compared to that of the group administered bevacizumab alone. Increased iNOS and decreased Arginase 1 are markers indicating an M1, immunostimulatory environment.

In addition, at day 20, post tumor injection, spleens were harvested and single cell suspensions were stained with mouse antibodies and analyzed by FACS. GMFI or frequency was calculated in Flowjo after gating on CD11b+F4/80+CD68+ cells (FIG. 17D-FIG. 17F). FIG. 17D and FIG. 17E show an increase in iNOS and a decrease of Arginase-1, respectively. Spleen cells were also stimulated overnight with 100 ng/ml lipopolysaccharide (LPS) and analyzed for TNF-α production. FIG. 17F shows an increase in TNF-α. Th1s data indicates a shift in splenic macrophages to an M1-like phenotype with IMPRIME PGG treatment. Arginase-1, iNOS and CD86 were analyzed in CD11b+Gr1+ cells and the data is shown in FIG. 17G-FIG. 171. As shown, Arginase-1 decreased and iNOS and CD86 increased, which indicates differentiation of splenic MDSC to M1/N1 macrophage/neutrophils with IMPRIME PGG treatment. FIG. 17J shows enhanced expression of co-stimulatory marker CD86 on tumor granulocytic MDSC (CD11b+Lyg6+) further indicating differentiation of splenic MDSC.

For FIG. 17K-FIG. 17M, tumors were harvested at day 20 and digested with type 1 collagenase for 1 hour at 37° C. Single cell suspensions were stained with mouse antibodies and analyzed by FACS. GMFI or frequency was calculated in Flowjo after gating on CD11b+ cells. As shown, PD-L1 and iNOS increased while Arginase-1 decreased indicating the shift in tumor-infiltrating myeloid cells to a M1-type phenotype. Th1s data clearly illustrates that soluble β-glucan increases the effectiveness of anti-angiogenic agents and modulates the TME, in vivo.

Lastly, single cell suspensions of the tumor cells were cultured overnight and supernatants were measured for TGF-β by ELISA The results are shown in FIG. 17N. Mice that showed greater than 50% tumor growth inhibition (TGI) when treated with a combination of IMPRIME PGG and bevacizumab, clearly showed a reduction in TGF-β.

As discussed previously, there is a subset of subjects that respond better to soluble β-glucan treatment. The results of FIG. 17N show that these subjects, who respond better to the combination treatment, would also have a concomitant reduction in TGF-β expression. Currently, TGF-β-inhibiting drugs, such as fresolimumab, are being studied as cancer therapy. As such, the subset of subjects that respond to soluble β-glucan would not require addition treatment with a TGF-β inhibitor. In addition, several methods and compositions have been described that transform non-responding subjects to subjects that respond to soluble β-glucan immunotherapy in International Patent Application. No. PCT/US2013/031625. By using these methods in combination with soluble β-glucan and bevacizumab treatment, all subjects would have reduced TGF-β expression, which would eliminate the need for additional treatment with a TGF-β inhibitor.

### Example 18

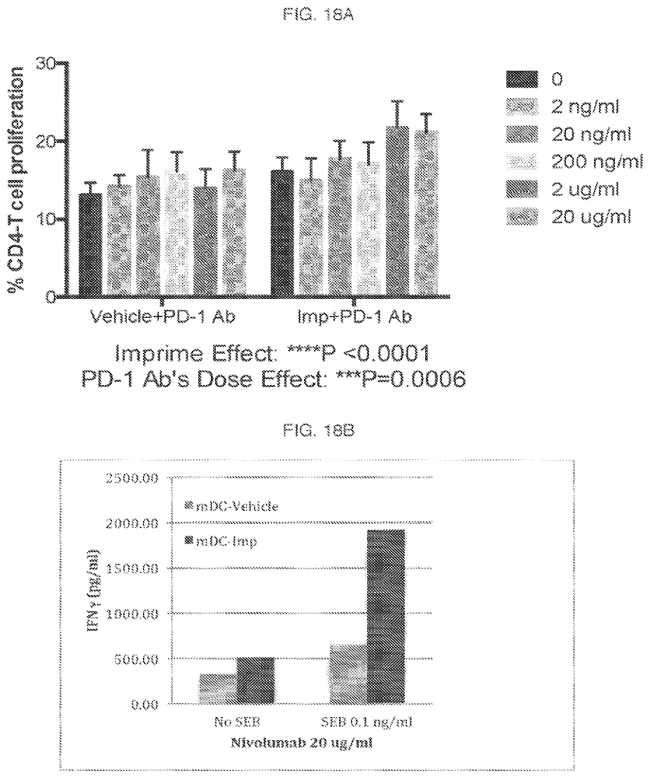

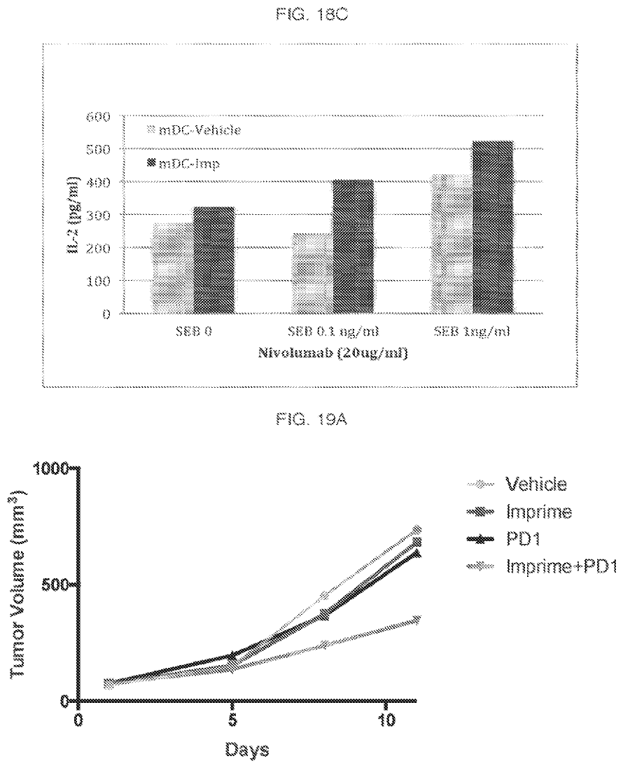

Soluble β-glucan in combination with anti-PD-1 antibodies affects tumors and the TME: In vitro studies were initially carried out to study the synergy between anti-human PD-1 Mab, nivolumab (Bristol Myers Squib) (a non-complement activating, non-tumor targeting antibody), and IMPRIME PGG The first study utilized an allogeneic-mixed lymphocyte reaction (MLR) using MoDC differentiated from IMPRIME PGG-treated monocytes Whole blood from a healthy donor was treated with IMPRIME PGG or vehicle for 2 hours at 37° C. Dendritic cells (DC) were generated by culturing monocytes isolated from PBMCs using negative selection with a monocyte purification kit (Thermo Fisher Scientific) in vitro for 7 days with 500 U/mL interleukin-4 (IL-4) and 250 U/mL GM-CSF (R&D Systems). CFSE labeled CD4⁺ T cells (1×10⁵) and allogeneic DCs (1×10⁴) were co-cultured with or without dose titrations of nivolumab or an isotype control antibody IgG4 added at the initiation of the assay. After 5 days, T-cell proliferation was measured by CFSE dilution assay and the results are shown in FIG. 18A. As indicated in the figure, especially at higher doses of nivolumab, treatment with IMPRIME PGG and nivolumab significantly increased T cell proliferation over treatment with antibody alone.

The second in vitro study looked at stimulation of PBMCs by the superantigen Staphylococcal enterotoxin B (SEB). Whole blood was treated with IMPRIME PGG or vehicle for 2 hours at 37° C. DCs were generated by culturing monocytes isolated from PBMCs using negative selection with a monocyte purification kit (Thermo Fisher Scientific) in vitro for 7 days with 500 U/mL interleukin-4 (IL-4) and 250 U/mL GM-CSF (R&D Systems). DCs were fully maturated by adding 25 ng/ml of TNFα and 50 ng/ml of LPS on day 5. PBMCs from a different donor was cultured with DCs at 10′1 ratio for 3 days with nivolumab or an isotype control antibody IgG₄ (20 ug/mL) at the initiation of the assay together with serial dilutions of SEB (Toxin Technology). IL-2 (BD Biosciences) and IFNγ (R&D Systems) levels in culture supernatants were measured by ELISA analysis and the results are shown in FIG. 18B and FIG. 18C. As indicated, the combination of nivolumab and IMPRIME PGG enhances T cell function including production of IFNγ and IL-2.

IL-2 acts on T cells and has been approved in the US and several European countries for treatment of cancers such as melanoma and renal cell cancer. However, administering IL-2 can have serious side effects. These data show that treatment with soluble β-glucan in combination with anti-PD-1 antibody increases IL-2 expression such that additional IL-2 treatment can be eliminated.

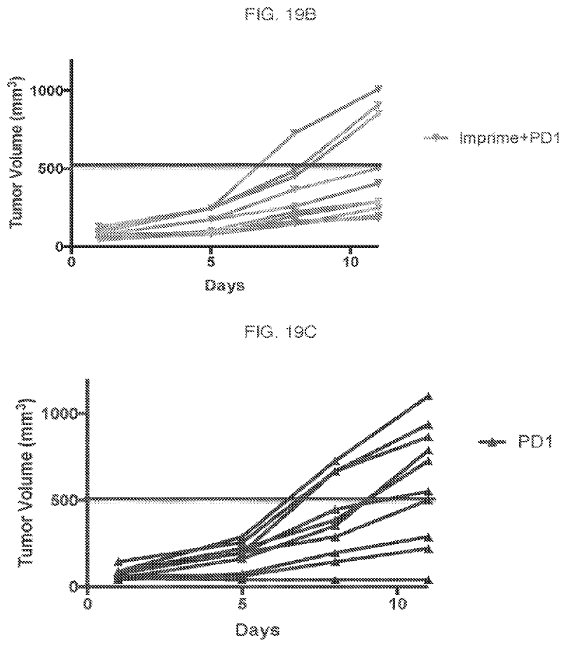

In vivo studies were carried out using IMPRIME PGG+anti-PD-1 antibody. IMPRIME PGG in combination with RMP1-14, an anti-PD-1 antibody, was tested in CT26 colon cancer-bearing C57B1/6 mice. 3×10⁵ mouse CT26 cells were administered subcutaneously. The mice were administered IMPRIME PGG (1.2 mg/mouse twice weekly I.V. for four weeks) alone, or anti-PD-1 antibody (200 μg/mouse twice weekly IP for three weeks) alone, or a combination of both after the tumors reached a size of 40-100 mm³. The results of the study are summarized in FIG. 19A.

The results show that the combination of IMPRIME PGG and anti-PD-1 antibody synergistically suppresses tumor growth in vivo. Th1s is even more evident from the individual mouse data shown in FIG. 19B (IMPRIME PGG+anti-PD-1 antibody) and FIG. 19C (anti-PD-1 antibody alone). 70% of mice treated with the combination therapy had tumors that remained under 500 mm³ while only 40% of mice treated only with anti-PD-1 antibody had tumors that remained under 500 mm³. Again, as discussed above, it seems that 30% of mice did not respond to soluble β-glucan immunotherapy, which skewed the overall results shown in FIG. 19A. However, transforming subjects to respond to soluble β-glucan immunotherapy could result in nearly all subjects showing increased tumor suppression with combination therapy.

### Example 19

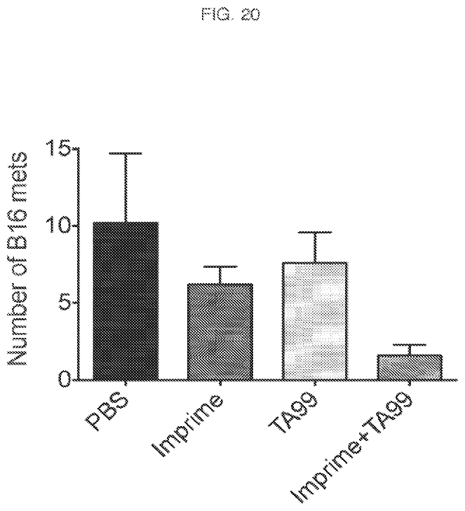

Soluble β-glucan therapy enhances in vivo anti-tumor efficacy of tumor-targeting antibodies in metastasized tumors: C57B1/6 mice were injected in the tail vein with B16 melanoma cells to generate experimental lung metastases. Four days later, mice were treated with IMPRIME PGG alone (1.2 mg/mouse twice weekly I.V. for three weeks), the anti-Trp1 antibody TA99 (50 pig/mouse twice weekly I.P. for three weeks) or the combination of both. At day 14, mice treated with the combination therapy of the tumor-specific antibody and IMPRIME PGG showed more than an 80% reduction in the outgrowth of these experimental metastases. The results are shown in FIG. 20.

### Example 20

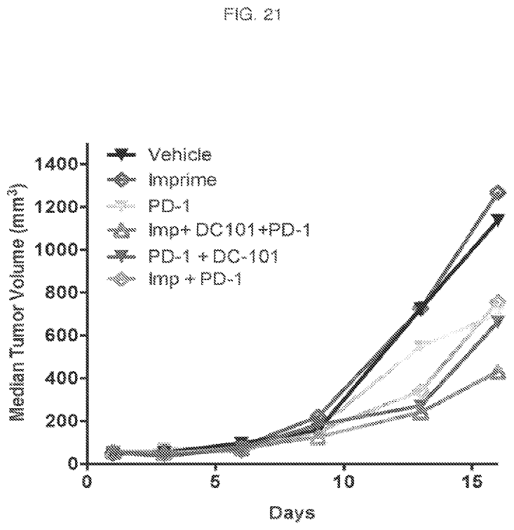

Soluble β-glucan therapy enhances in vivo the anti-tumor efficacy of the combination of anti-angiogenics and checkpoint inhibitors: IMPRIME PGG in combination with RMP1-14, an anti-PD-1 antibody and DC-101 was tested in CT26 colon cancer—bearing C57B1/6 mice. 3×10⁵ mouse CT26 cells were administered subcutaneously. The mice were administered IMPRIME PGG (1.2 mg/mouse twice weekly I.V. for four weeks), anti-PD-1 antibody (200 μg/mouse twice weekly I P. for four weeks), DC-101 (10 mg/kg i.v twice a week for four weeks) or combinations thereof after the tumors reached a size of 50-75 mm³ The results, shown in FIG. 21, show the combination of all three therapies was more effective than any of them alone or any combination of two therapies. Examples of other anti-angiogenics useful in this combination include, but are not limited to, bevacizumab and ranibizumab. In an alternative embodiment, an anti-tumor-targeting antibody may also be included in this therapy.

Thus, taken together, the data show that soluble β-glucan treatment in vivo can activate myeloid cells within both the tumor and spleen to orchestrate a profound shift in the TME, which promotes tumor recognition and suppression. Any of the combination therapies described herein may also include addition of a tumor-targeting antibody, which would be more effective due to the shift in the TME.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters set forth herein are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A composition for use in immunotherapy comprising:
a soluble β-glucan;
a checkpoint inhibitor; and
an anti-angiogenic antibody.

2. The composition of claim 1, wherein the checkpoint inhibitor is one of either an anti-PD-1 antibody or an anti-PD-L1 antibody.

3. The composition of claim 2, wherein the anti-PD-L1 antibody is a non-complement-activating antibody.

4. The composition of claim 2, wherein the anti-PD-L1 antibody is an Fc-engineered IgG₁ antibody.

5. The composition of claim 1, wherein the soluble β-glucan is soluble β(1,6)-[poly-(1,3)-D-glucopyranosyl]-poly-β(1,3)-D-glucopyranose.

6. The composition of claim 1, wherein the soluble β-glucan is derived from yeast.

7. The composition of claim 6, wherein the yeast is Saccharomyces cerevisiae.

8. The composition of claim 1, wherein the soluble β-glucan, the checkpoint inhibitor, and the anti-angiogenic antibody are in a single formulation.

9. The composition of claim 1, wherein the soluble β-glucan, the checkpoint inhibitor, and the anti-angiogenic antibody are in separate formulations.

10. The composition of claim 1, wherein the anti-angiogenic antibody is an anti-VEGFR2 antibody.

11. The composition of claim 1, wherein the anti-angiogenic antibody is an anti-VEGFR antibody.

12-17. (canceled)
